(19) United States
(12) Patent Application Publication
(10) Pub. No.: US 2015/0024398 A1
(43) Pub. Date: Jan. 22, 2015

(54) ANALYSIS OF GENETIC BIOMARKERS FOR FORENSIC ANALYSIS AND FINGERPRINTING

(71) Applicant: Nina M. Hofstadler, Vista, CA (US)

(72) Inventors: Stanley Motley, Carlsbad, CA (US); Kristin Sannes-Lowery, Irvine, CA (US); Mark W. Eshoo, San Diego, CA (US); Steven A. Hofstadler, Vista, CA (US)

(73) Assignee: IBIS BIOSCIENCES, INC., Carlsbad, CA (US)

(21) Appl. No.: 14/237,251

(22) PCT Filed: Aug. 3, 2012
(86) PCT No.: PCT/US12/49589

§ 371 Date: Sep. 18, 2014

Related U.S. Application Data
(60) Provisional application No. 61/515,688, filed on Aug. 5, 2011.

(51) Int. Cl. C12Q 1/68; C12Q 1/70

(52) U.S. Cl. CPC C12Q 1/686; C12Q 1/6872; C12Q 1/689; C12Q 1/701; C12Q 1/6895
USPC 435/6.12

(57) ABSTRACT

The present invention relates generally to methods of determining base compositions for PCR products (e.g., RT PCR products, (rt) RT-PCR products, etc.). In particular, the present invention provides base-composition determination of PCR products containing up to five different nucleobases (e.g., A, C, G, T, U) and/or significant levels of non-templated adenylation.

### CROSS-REFERENCE TO RELATED APPLICATION

The present Application claims priority to U.S. Provisional Application Ser. No. 61/515,688 filed Aug. 5, 2011, the entirety of which is herein incorporated by reference.

The invention was made, in part, using funds from HSARPA Grant #NBCHC070041 and DHS Grant #N10PC20100. The government has certain rights in the invention.

### FIELD OF THE INVENTION

The present invention relates generally to methods of determining base compositions for PCR products (e.g., RT PCR products, (rt) RT-PCR products, etc.). In particular, the present invention provides base-composition determination of PCR products containing up to five different nucleobases (e.g., A, C, G, T, U) and/or significant levels of non-templated adenylation.

### BACKGROUND OF THE INVENTION

Nucleic acid signatures are commonly used for the detection and tracking of pathogens in many fields, including microbial forensics. Biological or environmental samples may contain viruses, bacteria, and/or eukaryotic cells that require identification. Depending on the organism of interest, either DNA or RNA detection may be appropriate.

Broad range polymerase chain reaction followed by electrospray ionization mass spectrometry (PCR/ESI-MS) is a rapid, high-throughput method for identification, characterization, and/or quantification of microorganisms including bacteria, virus and fungi (Ecker, et al., Proc Nail Acad Sci USA. 102, 8012-8017 2005; Ecker, et al., Nat Rev Microbiol, 6, 553-558 2008; Massire, et al., J Clin Microbiol, 49, 908-917 2011; herein incorporated by reference in their entireties). The PCR/ESI-MS technique identifies microorganisms by determining the precise molecular mass of the individual strands of the PCR products followed by bioinformatic triangulation based on the calculated unambiguous base compositions of those products.

Real-time polymerase chain reaction (RT-PCR), also called quantitative real time polymerase chain reaction (Q-PCR/qPCR/qrt-PCR) or kinetic polymerase chain reaction (KPCR), is a PCR-based technique used to simultaneously amplify and quantify a target nucleic acid molecule. RT-PCR and reverse transcription Real-time polymerase chain reaction ((rt) RT-PCR) offer the sensitivity and specificity necessary for correct identification of trace levels of the organisms of interest (McAvin, et al., J Clin Microbiol. 39, 3446-3451 2001; Verstrepen, et al., J Clin Virol, 25 Suppl J, 539-43 2002; Wellinghausen, et al., Appl Environ Microbiol, 67, 3985-3993 2001; herein incorporated by reference in their entireties). The use of either technique requires significant effort to prevent sample contamination as well as the inclusion or positive and negative controls to provide confidence in the accuracy of a given detection required for microbial forensics. The use of positive controls is essential to ensure the target of interest will be detected with the assay conditions used, although it adds the risk of carryover contamination to the test sample. Synthetic templates are a typical choice for positive controls, and usually are constructed to contain a small insert or deletion in a region outside the primer or probe binding regions of the target sequence (Mackay, et al., J Clin Virol. 28, 291-302 2003; herein incorporated by reference in its entirety). Positive controls are indistinguishable from positive samples based solely on the cycle threshold (Ct) values obtained from a typical RT-PCR reaction. Therefore any sample contamination with the positive control, or carry-over contamination, could result in a false positive detection. Contamination from carry-over products is a recognized problem for RT-PCR and similar techniques (Kwok, PCR Protocols (Innis et al. Academic Press 1990), Chapter 17, pages 142-145.; incorporated herein by reference in its entirety). One method of controlling for carry-over contamination in RT-PCR reactions is the incorporation of uracils in place of thymines, combined with uracil N-glycosylase (UNG) treatment to digest any residual RT-PCR products (Pang, et al., Mol Cell Probes. 6, 251-256 1992.; U.S. Pat. No. 5,418,149; herein incorporated by reference in their entireties). The use of deoxyuridine (in the form of dUTP) in the reactions results in products containing combinations of five different nucleotides: adenosines, thymidines, guanosines, cytidines and uridines, since the primers contain thymines and the polymerase incorporates uracils. The presence of five different nucleotides in the reaction products complicates determining the identity of the products. Additionally, the specific polymerase may incorporate non-templated adenosines (Smith, et al., Genome Res, 5, 312-317 1995), further complicating the analysis.

### SUMMARY OF THE INVENTION

The present invention is directed towards methods of determining base compositions for PCR products (e.g., RT PCR products, (rt) RT-PCR products, etc.). In some embodiments, the present invention provides base-composition determination of amplicons containing (or potentially containing) five different nucleobases (e.g., A, C, G, T, U). In some embodiments, the present invention provides base-composition determination of amplicons containing (or potentially containing) non-templated adenylation.

In some embodiments, the present invention is directed towards methods of identifying a bioagent, organism, and/or pathogen in a sample (e.g., biological and/or environmental) by obtaining nucleic acid from a biological sample, selecting at least one pair of primers with the capability of amplification of nucleic acid of the bioagent, organism, and/or pathogen, amplifying the nucleic acid (e.g., by RT PCR products, (rt) RT-PCR, qPCR, etc.) with the primers to obtain at least one amplification product, and determining the molecular mass of at least one amplification product from which the bioagent, organism, and/or pathogen is identified.

In some embodiments, the present invention provides a method of detecting the presence of a nucleic acid in a sample comprising: (a) enzymatically amplifying a segment of the nucleic acid to produce an amplicon comprising five or more different types of nucleotides; (b) measuring the molecular mass of the amplicon by mass spectrometry; and (c) determining a base composition of the amplicon, (d) detecting the presence of the nucleic acid in the sample. In some embodiments, enzymatically amplifying comprises amplifying by PCR. In some embodiments, amplifying by PCR comprises amplifying by RT-PCR, (rt) RT-PCR, or qPCR. In some embodiments, enzymatically amplifying comprises combining the nucleic acid or the segment thereof in a reaction vessel with: (i) a primer pair comprising a forward primer and a reverse primer, (ii) a mixture of conventional dNTPs, wherein the mixture is lacking one dNTP selected from dATP, dCTP, dGTP, or dTTP; (iii) a modified dNTP; (iv) a DNA polymerase enzyme capable of incorporating the modified dNTP in place of the dNTP missing from the mixture of conventional dNTPs; and (v) appropriate buffer, salt and pH conditions for enzymatic amplification of nucleic acid. In some embodiments, the method further comprises a step before step (a) of treating the reaction vessel with an enzyme that cleaves DNA molecules at the modified dNTP. In some embodiments, the dNTP missing from the mixture of conventional dNTPs is dTTP. In some embodiments, the modified dNTP is dUTP. In some embodiments, the method comprises a step before step (a) of treating the reaction vessel with uracil N-glycosylase. In some embodiments, the primers bind to conserved regions of the nucleic acid, wherein the conserved regions of the nucleic acid flank a variable region of the nucleic acid. In some embodiments, the base composition of the variable region is sufficient to identify the genus, species, and/or strain of the bioagent from which the nucleic acid was obtained. In some embodiments, the primers do not comprise the modified nucleotide. In some embodiments, the primers comprise deoxyadenosine, deoxycytidine, deoxyguanosine, and deoxythymidine. In some embodiments, the amplicon comprises deoxyadenosine, deoxycytidine, deoxyguanosine, deoxythymidine, and deoxyuridine. In some embodiments, mass spectrometry comprises ESI-MS. In some embodiments, determining a base composition of the amplicon does not comprise determining the sequential order of nucleotides in the amplicon (i.e., the number of each nucleotide present is identified, e.g., A₁₂T₁₀C₅G₉U₃, without identifying the linear sequence of the nucleotides). In some embodiments, methods described herein prevent carryover contamination.

In some embodiments, the present invention provides a method of detecting the presence of a nucleic acid comprising: (a) combining the nucleic acid or a portion thereof in a reaction vessel with: (i) a primer pair comprising a forward primer and a reverse primer, (ii) a mixture of conventional dNTPs, wherein the mixture is lacking one dNTP selected from dATP, dCTP, dGTP, or dTTP; (iii) a modified dNTP; (iv) a DNA polymerase enzyme capable of incorporating the modified dNTP in place of the dNTP missing from the mixture of conventional dNTPs; and (v) an enzyme that cleaves DNA molecules at the modified dNTP; (b) incubating the contents of the reaction mixture at a temperature wherein the enzyme that cleaves DNA molecules at the modified dNTP is active, but the DNA polymerase enzyme is not active, under conditions and for a time sufficient to degrade nucleic acids containing the modified dNTP; (c) incubating the contents of the reaction mixture at a temperature wherein the DNA polymerase enzyme is active, but the enzyme that cleaves DNA molecules at the modified dNTP is not active, under conditions and for a time sufficient to amplify a segment of the nucleic acid to produce an amplicon; (d) measuring the molecular mass of the amplicon by mass spectrometry; and (e) determining a base composition of the amplicon, and detecting the presence of the nucleic acid as comprising a segment with a base composition corresponding to the base composition of the amplicon. In some embodiments, the dNTP missing from the mixture of conventional dNTPs is dTTP. In some embodiments, the modified dNTP is dUTP. In some embodiments, the enzyme that cleaves DNA molecules at the modified dNTP is uracil N-glycosylase. In some embodiments, the primers bind to conserved regions of the nucleic acid, wherein the conserved regions of the nucleic acid flank a variable region of the nucleic acid. In some embodiments, the base composition of the variable region is sufficient to identify the genus, species, and/or strain of the bioagent from which the nucleic acid was obtained. In some embodiments, the primers do not comprise the modified nucleotide. In some embodiments, the primers comprise deoxyadenosine, deoxycytidine, deoxyguanosine, and deoxythymidine. In some embodiments, the amplicon comprises deoxyadenosine, deoxycytidine, deoxyguanosine, deoxythymidine, and deoxyuridine. In some embodiments, mass spectrometry comprises ESI-MS. In some embodiments, determining a base composition of the amplicon does not comprise determining the sequential order of nucleotides in the amplicon. In some embodiments, the DNA polymerase is a thermostable DNA polymerase. In some embodiments, determining a base composition comprises correcting the molecular weight contribution of the modified dNTPs with a molecular weight contribution for a corresponding number of the dNTP missing from the mixture of conventional dNTPs. In some embodiments, the enzyme that cleaves DNA molecules at the modified dNTP is active at a temperature range between 45 and 60° C. In some embodiments, the enzyme that cleaves DNA molecules at the modified dNTP is not active, or minimally active, above a temperature of 60° C.

In some embodiments, the present invention provides a method of detecting the presence of a nucleic acid comprising: (a) amplifying a segment of the nucleic acid with an amplification enzyme to produce amplicons, wherein the amplification enzyme catalyzes non-templated adenylation; (b) measuring the molecular mass of the amplicon by mass spectrometry; (c) determining a base composition of the template portion of the amplicon by correcting for the incorporation of non-templated adenylation; (e) detecting the presence of the nucleic acid. In some embodiments, the mass spectrometry comprises ESI-MS. In some embodiments, determining a base composition of the amplicon does not comprise determining the sequential order of nucleotides in the amplicon. In some embodiments, amplifying comprises amplifying by PCR. In some embodiments, amplifying by PCR comprises amplifying by RT-PCR, (rt) RT-PCR, or qPCR. In some embodiments, the amplification enzyme comprises a DNA polymerase.

In some embodiments, the present invention provides a method of detecting the presence of a nucleic acid in a sample comprising: (a) combining the nucleic acid or a portion thereof in a reaction vessel with: (i) a primer pair comprising a forward primer and a reverse primer, (ii) a mixture of conventional dNTPs, wherein the mixture is lacking one dNTP selected from dATP, dCTP, dGTP, or dTTP; (iii) a modified dNTP; (iv) a DNA polymerase enzyme capable of incorporating the modified dNTP in place of the dNTP missing from the mixture of conventional dNTPs, wherein the DNA polymerase enzyme is capable of catalyzing non-templated adenylation; and (v) an enzyme that cleaves DNA molecules at the modified dNTP; (b) incubating the contents of the reaction mixture at a temperature wherein the enzyme that cleaves DNA molecules at the modified dNTP is active, but the DNA polymerase enzyme is not active, under conditions and for a time sufficient to degrade nucleic acids containing the modified dNTP; (c) incubating the contents of the reaction mixture at a temperature wherein the DNA polymerase enzyme is active, but the enzyme that cleaves DNA molecules at the modified dNTP is not active, under conditions and for a time sufficient to amplify a segment of the nucleic acid to produce an amplicon; (d) measuring the molecular mass of the amplicon by mass spectrometry; (e) determining a base composition of the amplicon by correcting for the incorporation of non-templated adenylation; and (f) detecting the presence of the nucleic acid in a sample. In some embodiments, the dNTP missing from the mixture of conventional dNTPs is dTTP. In some embodiments, the modified dNTP is dUTP. In some embodiments, the enzyme that cleaves DNA molecules at the modified dNTP is uracil N-glycosylase. In some embodiments, the primers bind to conserved regions of the nucleic acid, wherein the conserved regions of the nucleic acid flank a variable region of the nucleic acid. In some embodiments, the base composition of the variable region is sufficient to identify the genus, species, and/or strain of the bioagent from which the nucleic acid was obtained. In some embodiments, the primers do not comprise the modified nucleotide. In some embodiments, the primers comprise deoxyadenosine, deoxycytidine, deoxyguanosine, and deoxythymidine. In some embodiments, the amplicon comprises deoxyadenosine, deoxycytidine, deoxyguanosine, deoxythymidine, and deoxyuridine. In some embodiments, mass spectrometry comprises ESI-MS. In some embodiments, determining a base composition of the amplicon does not comprise determining the sequential order of nucleotides in the amplicon. In some embodiments, the DNA polymerase is a thermostable DNA polymerase. In some embodiments, determining a base composition comprises correcting the molecular weight contribution of the modified dNTPs with a molecular weight contribution for a corresponding number of the dNTP missing from the mixture of conventional dNTPs. In some embodiments, the enzyme that cleaves DNA molecules at the modified dNTP is active at a temperature range between 45 and 60° C. In some embodiments, the enzyme that cleaves DNA molecules at the modified dNTP is not active, or minimally active, above a temperature of 60° C.

### BRIEF DESCRIPTION OF THE DRAWINGS

FIG. 1 shows representative ESI-MS mass spectra for (A) (rt) RT-PCR (Flexal Virus) and (B) RT-PCR (B. melitensis). For each target, panel A and B are drawn to scale to illustrate the shift in molecular weight of the isolate and CST Real-Time PCR products. Arrows indicate both forward and reverse strands with base composition shown for the forward strand.

FIG. 2 shows SNP identification for the C. botulinum isolate. Identification of SNP by ESI-MS basecount determination (A); SNP confirmation by DNA sequence analysis (B). Isolate sequence was compared to GenBank reference sequence for C. botulinum F (Accession No. CP000728.1).

FIG. 3 shows representative mass spectra and base compositions (A G C T/U) of the B6 amplicons. Non-adenylated and adenylated forms were identified for both strands of the B6-WT and B6-MT amplicons. Arrows indicate the nonadenylated and adenylated forms of each strand.

FIG. 4 shows representative Mass spectra and base compositions (A G C T/U) of the R2 amplicons. Non-adenylated and adenylated forms of both strands of R2-WT and R2-MT amplicons were identified.

FIG. 5 shows representative Mass spectra and base compositions (A G C T/U) of the A7 amplicons. Non-adenylated and adenylated forms of both strands were observed for the WT and MT amplicons.

FIG. 6 shows representative mass spectra and base compositions (A G C T/U) of the re-PCR products of the L4 amplicons. L4 real-time PCR amplicons were amplified in a second round of PCR according to methods described herein, then analyzed. Non-adenylated and adenylated forms were observed. The base compositions reflect the use of A-tailed primers in the secondary PCR amplification.

FIG. 7 shows representative ESI-MS mass spectra for reverse transcription RT-PCR (Flexal Virus) and RT-PCR (B. melitensis). Flexal virus and B. melitensis isolate (A); Flexal virus and B. melitensis CST (B).

FIG. 8 shows representative ESI-MS mass spectra of isolate and CST from reaction mixtures containing both isolate and CST templates during reverse transcription RT-PCR (Flexal Virus) and RT-PCR (R. rickettsii). Reaction mixtures contained an excess of isolate template over CST with approximately 100:10 copies isolate:CST for Flexal virus (A) and approximately 10,000:100 copies for R. rickettsii (B).

FIG. 9 shows SNP identification for the C. botulinum isolate. Identification of SNP by ESI-MS base count determination (A); SNP confirmation by DNA sequence analysis (B). Isolate sequence was compared to GenBank reference sequence for C. botulinum F (Accession CP000728.1).

### DESCRIPTION OF EMBODIMENTS

The present invention relates generally to methods of determining base compositions for PCR products (e.g., RT PCR products, (rt) RT-PCR products, etc.) or other amplification products or other synthesized nucleic acid molecules. In particular, the present invention provides base-composition determination of PCR products containing, for example, up to five different nucleobases (e.g., A, C, G, T, U) and/or non-templated adenylation. In some embodiments, base-composition is determined for amplicons comprising more than four different types of nucleotides (e.g., 5 (e.g., A, C, G, T, U), 6, 7, 8, 9, 10, or more). In some embodiments, base-composition is determined for amplicons comprising non-templated nucleotides (e.g., non-templated adenylation). In some embodiments, base compositions are determined, correcting for the presence of both uridine and thymidine in an amplicon (e.g., converting one to the other). In some embodiments, base compositions are determined, correcting for the presence of non-templated adenylation. The present method provides rapid throughput and does not require nucleic acid sequencing of the amplified target sequence for bioagent detection and identification.

Reverse transcription RT-PCR is a useful technique for microbial forensics due to its ability to detect low levels of specific biological agents, including bacterial, viral and eukaryotic targets. Positive controls are often essential to demonstrate successful amplification with the designated primers, probes, and reaction conditions each time the assay is performed. Typically, a positive control template is identical to a test sample with a small sequence variation, such as an insertion or deletion of several bases. When testing an unknown sample it is important to establish that a positive result was not due to contamination by the positive control. Individual (rt) RT-PCR reactions alone are not capable of distinguishing between a true positive and a false positive arising from contamination with positive control. However, a true positive will differ in sequence and molecular weight from the positive control and therefore can be differentiated.

DNA sequence analysis of the product has historically been required for the confirmation of a positive (rt) RT-PCR result. Such analysis is both time consuming and problematic for short products without additional molecular manipulation. The methods of the present invention provide a rapid alternative means (e.g., using ESI-MS), without additional manipulation, of confirmation within a short timeframe (e.g., less than an hour) following the identification of a potential positive.

In some embodiments, the molecular weights of the forward and reverse strands of the (rt) RT-PCR products are determined by the ESI-MS. In some embodiments, both the forward and reverse strands of the (rt) RT-PCR products generate a MS peak relating specific molecular weight. In some embodiments, the base composition is determined from the precise molecular mass determination of the forward and reverse strand for each product (Ecker et al. (2008) Nat Rev Microbiol, 6, 553-558.; Sampath (2005) Emerg Inject Dis, 11, 373-379.; herein incorporated by reference in its entirety). In some embodiments, the difference between a true positive and a positive control is determined from the mass spectrum, molecular weight, and/or base composition. In some embodiments, differing molecular weights of amplicons are reflected in unique base compositions.

Experiments conducted during development of embodiments of the present invention demonstrated equivalent sensitivity between (rt) RT-PCR detection and ESI-MS detection of the same products. Products from both RT-PCR and (rt) RT-PCR reaction chemistries were successfully identified by ESI-MS. In some cases, ESI-MS demonstrated greater sensitivity, detecting positive samples that were negative by (rt) RT-PCR (i.e., undetermined Ct value). For example, methods of the present invention successfully detected products from bacterial, viral and plant nucleic acids from organisms of forensic interest at very low levels, and distinguished the products from their respective positive controls.

In some embodiments, the molecular weight as determined by ESI-MS methods described herein are capable of detecting otherwise unidentified SNPs in samples. For example, experiments conducted during development of embodiments of the present invention demonstrated the detection of an unidentified SNP in the C. botulinum F isolate compared to the reference sequence reported in GenBank. The SNP was not likely due to polymerase error during RT-PCR as it was identified in each of the multiple replicates tested. The SNP was confined by sequencing analysis, which identified the specific G to A transition predicted by the ESI-MS analysis.

In some embodiments, methods described herein are capable of detecting multiple base differentials between isolates and positive controls as well as a single base SNP in one of the isolates. In some embodiments, methods of the present invention have broad applicability for quality control of (rt) RT-PCR reactions. In addition to identifying PCR products and RT-PCR products (Chen, et al., Diagn Microbiol Infect Dis, 69, 179-186 2011; Ecker, et al., Nat Rev Microbiol, 6, 553-558 2008; herein incorporated by reference in their entireties), methods described herein are capable of determining base compositions and thereby identifying the products of RT-PCR reactions containing five different nucleotides (e.g., A, C, G, T, and U) by ESI-MS, as demonstrated by experiments conducted during development of embodiments of the present invention.

Further experiments conducted during the course of development of embodiments of the present invention demonstrated equivalent sensitivity between RT-PCR or reverse transcriptase RT-PCR detection and ESI-MS detection of the same products. Products from both reaction types were successfully identified by ESI-MS, and the ESI-MS was able to detect positive samples that were negative by RT-PCR or reverse transcriptase RT-PCR (undetermined Ct value). Products from bacterial, viral and plant nucleic acids from organisms of forensic interest were successfully detected at very low levels and were distinguished from their respective positive controls.

The ability of ESI-MS analysis to identify CST contamination in an isolate sample was demonstrated for both RT-PCR and reverse transcriptase RT-PCR conditions. While both templates contributed to the Ct value, it was only with the ESI-MS analysis of the reaction products that the contribution from both templates was identified. The CST contamination level was clearly identified even though it was a minor constituent of the reaction template.

Additionally, the molecular weight as determined by ESI-MS indicated a potential SNP in the C. botulinum F isolate that was evaluated compared to the reference sequence reported in GenBank. The SNP was not likely due to polymerase error during RT-PCR as it was identified in each of the multiple replicates tested. The SNP was confirmed by sequencing analysis, which identified the specific G to A transition predicted by the ESI-MS analysis.

The ESI-MS successfully detected multiple base differentials between isolates and positive controls as well as a single base SNP in one of the isolates. The technique has broad applicability for example, in quality control of RT-PCR and reverse transcriptase RT-PCR reactions. In addition to previously reported capabilities identifying PCR products and RT-PCR products (Ecker et al., Nat Rev Microbiol 2008; 6(7):553-8; Chen et al., Diagn Microbiol Infect Dis 2011; 69(2):179-86), experiments described herein demonstrated the successful identification of RT-PCR reactions containing five different nucleotides (including uracils) by ESI-MS.

Verification of positive results is important so that forensic scientists, policymakers and law enforcement are confident in the detection of biothreat agents. The ESI-MS method allows the use of the exact same primers and probes to eliminate ambiguity that may arise from the use of alternative primers, probes or detection methods to discriminate controls from test samples. Other methods such as melting curve analysis are incompatible with probe based RT-PCR detection such as those used herein, and the use of additional qPCR probe(s) introduces new variables, requiring a completely separate validation performed in a restrictive bio-containment environment while not guaranteeing equivalence in sensitivity or specificity. The ESI-MS method provides policy makers with a definitive determination that the detected signal originates from a true biological presence rather than the positive control.

The present invention provides, inter alia, methods for characterization, detection, and identification of nucleic acids in a sample. In some embodiments, nucleic acids for analysis by the methods herein are from any source (e.g., biological, clinical, research, synthetic, environmental) and are analyzed for any purpose (e.g., bioagent detection, diagnosis, research, etc.). In some embodiments, nucleic acids from one or more bioagents are identified (thereby identifying and/or detecting one or more bioagents in a sample) in an unbiased manner using “bioagent identifying amplicons.” In some embodiments, nucleic acids in a sample are amplified by PCR or a related technique (e.g., RT-PCR, q-PCR, (rt) RT-PCR, etc.), and the mass of the resulting amplicon(s) are determined by methods described herein (e.g., mass spectrometry (e.g., ESI-MS)). In some embodiments, base compositions are determined from the mass of amplicons by methods described herein. In some embodiments, base compositions are used to identify the source (e.g., bioagent) of an amplicon. In some embodiments, methods are provided herein for determining masses and base compositions for amplicons (e.g., produced by PCR or a related technique (e.g., RT-PCR, q-PCR, (rt) RT-PCR, etc.)) containing up to five different nucleotides (e.g., A, C, G, T, U). In some embodiments, methods are provided for mass and base composition determination of amplicons containing non-templated adenylation (e.g., substantial or high levels of non-templated adenylation). In some embodiments, methods are provided for differentiating test amplicons (e.g., containing up to 5 different nucleotides) from control nucleic acids (e.g., containing up to 5 different nucleotides). In some embodiments, methods provide a means for eliminating carry-over contamination, and problems associated therewith.

As used herein, the term “carryover contamination” refers to nucleic acid molecules inadvertently present in an amplification reaction that are suitable templates for amplification by primers in the amplification reaction. Carryover typically occurs from aerosol or other means of physically transferring amplified product generated from earlier amplification reactions into a different, later, amplification reaction. Carryover contamination may also result from traces of nucleic acid which originate with the amplification reagents. Carryover contamination commonly occurs as the result of positive control molecules contaminating subsequent amplification reactions.

In the context of this invention, a “bioagent” is any organism, cell, or virus, living or dead, or a nucleic acid derived from such an organism, cell or virus. Examples of bioagents include, but are not limited, to cells (including, but not limited to, human clinical samples, bacterial cells and other pathogens) viruses, fungi, and protists, parasites, and pathogenicity markers (including, but not limited to, pathogenicity islands, antibiotic resistance genes, virulence factors, toxin genes and other bioregulating compounds). Samples may be alive or dead or in a vegetative state (for example, vegetative bacteria or spores) and may be encapsulated or bioengineered. Samples may be forensic samples. In the context of this invention, a “pathogen” is a bioagent that causes a disease or disorder.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. Environmental samples include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to the present invention.

Despite enormous biological diversity, all forms of life on earth share sets of essential, common features in their genomes. Bacteria, for example, have highly conserved sequences in a variety of locations on their genomes. Most notable is the universally conserved region of the ribosome, but there are also conserved elements in other non-coding RNAs, including RNAse P and the signal recognition particle (SRP) among others. Bacteria have a common set of absolutely required genes. About 250 genes are present in all bacterial species (Mushegian et al., Proc. Natl. Acad. Sci. U.S.A., 1996, 93, 10268; and Fraser et al., Science, 1995, 270, 397), including tiny genomes like Mycoplasma, Ureaplasma and Rickettsia. These genes encode proteins involved in translation, replication, recombination and repair, transcription, nucleotide metabolism, amino acid metabolism, lipid metabolism, energy generation, uptake, secretion and the like. Examples of these proteins are DNA polymerase III beta, elongation factor TU, heat shock protein groEL, RNA polymerase beta, phosphoglycerate kinase, NADH dehydrogenase, DNA ligase, DNA topoisomerase and elongation factor G. Operons can also be targeted using the present method. One example of an operon is the bfp operon from enteropathogenic E. coli. Multiple core chromosomal genes can be used to classify bacteria at a genus or genus species level to determine if an organism has threat potential. The methods can also be used to detect pathogenicity markers (plasmid or chromosomal) and antibiotic resistance genes to confirm the threat potential of an organism and to direct countermeasures.

Since genetic data provide the underlying basis for identification of bioagents by the methods of the present invention, it is prudent to select segments of nucleic acids which ideally provide enough variability to distinguish each individual bioagent and whose molecular mass is amenable to molecular mass determination. In one embodiment of the present invention, at least one polynucleotide segment is amplified to facilitate detection and analysis in the process of identifying the bioagent. Thus, the nucleic acid segments that provide enough variability to distinguish each individual bioagent and whose molecular masses are amenable to molecular mass determination are herein described as “bioagent identifying amplicons.” The term “amplicon” as used herein, refers to a segment of a polynucleotide which is amplified in an amplification reaction (e.g., PCR, RT-PCR, (rt) RT-PCR, qPCR, etc.). In some embodiments of the present invention, bioagent identifying amplicons comprise from about 45 to about 150 nucleobases (i.e. from about 45 to about 150 linked nucleosides). One of ordinary skill in the art will appreciate that the invention embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleobases in length.

As used herein, “intelligent primers” are primers that are designed to bind to highly conserved sequence regions that flank an intervening variable region and yield amplification products which ideally provide enough variability to distinguish each individual bioagent, and which are amenable to molecular mass analysis. By the term “highly conserved,” it is meant that the sequence regions exhibit between about 80-100%, or between about 90-100%, or between about 95-100% identity. The molecular mass of a given amplification product provides a means of identifying the bioagent from which it was obtained, due to the variability of the variable region. Thus, design of intelligent primers involves selection of a variable region with appropriate variability to resolve the identity of a particular bioagent. It is the combination of the portion of the bioagent nucleic acid molecule sequence to which the intelligent primers hybridize and the intervening variable region that makes up the bioagent identifying amplicon. Alternately, it is the intervening variable region by itself that makes up the bioagent identifying amplicon.

It is understood in the art that the sequence of a primer need not be 100% complementary to that of its target nucleic acid to be specifically hybridizable. Moreover, a primer may hybridize over one or more segments such that intervening or adjacent segments are not involved in the hybridization event (e.g., a loop structure or hairpin structure). The primers of the present invention can comprise at least 70%, at least 75%, at least 80%, at least 85%, at least 90%, at least 95%, or at least 99% sequence complementarity to the target region within the highly conserved region to which they are targeted. For example, an intelligent primer wherein 18 of 20 nucleobases are complementary to a highly conserved region would represent 90 percent complementarity to the highly conserved region. In this example, the remaining noncomplementary nucleobases may be clustered or interspersed with complementary nucleobases and need not be contiguous to each other or to complementary nucleobases. As such, a primer which is 18 nucleobases in length having 4 (four) noncomplementary nucleobases which are flanked by two regions of complete complementarity with the highly conserved region would have 77.8% overall complementarity with the highly conserved region and would thus fall within the scope of the present invention. Percent complementarity of a primer with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

In some embodiments, primers for use in embodiments of the present invention comprise up to four different types of nucleobases (e.g., A, C, G, T). In some embodiments, primers do not contain uridine nucelobases (e.g., UTP). In some embodiments, primers lack a nucleobase that is present as a component of the amplification reaction (e.g. uridine). In some embodiments, primers comprise a nucleobase (e.g., uridine) that is otherwise present as a component of the amplification reaction (e.g. thymidine).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, complementarity of intelligent primers, is between about 70% and about 80%. In other embodiments, homology, sequence identity or complementarity, is between about 80% and about 90%. In yet other embodiments, homology, sequence identity or complementarity, is about 90%, about 92%, about 94%, about 95%, about 96%, about 97%, about 98%, about 99% or about 100%.

In some embodiments, intelligent primers comprise from about 12 to about 35 nucleobases (i.e. from about 12 to about 35 linked nucleosides). One of ordinary skill in the art will appreciate that the invention embodies compounds of 12, 13, 14, 15, 16, 17, 18, 19, 20, 21, 22, 23, 24, 25, 26, 27, 28, 29, 30, 31, 32, 33, 34, or 35 nucleobases in length.

One having skill in the art armed with the preferred bioagent identifying amplicons defined by the primers illustrated herein will be able to identify additional intelligent primers.

Bioagent identifying amplicons may be found in any region of a given genome, wherein the nucleic acid sequence meets the above identified criteria for producing a bioagent identifying amplicon. In one embodiment, the bioagent identifying amplicon is a portion of a ribosomal RNA (rRNA) gene sequence. With the complete sequences of many of the smallest microbial genomes now available, it is possible to identify a set of genes that defines “minimal life” and identify composition signatures that uniquely identify each gene and organism. Genes that encode core life functions such as DNA replication, transcription, ribosome structure, translation, and transport are distributed broadly in the bacterial genome and are suitable regions for selection of bioagent identifying amplicons. Ribosomal RNA (rRNA) genes comprise regions that provide useful base composition signatures. Like many genes involved in core life functions, rRNA genes contain sequences that are extraordinarily conserved across bacterial domains interspersed with regions of high variability that are more specific to each species. The variable regions can be utilized to build a database of base composition signatures. The strategy involves creating a structure-based alignment of sequences of the small (16S) and the large (23S) subunits of the rRNA genes. For example, there are currently over 13,000 sequences in the ribosomal RNA database that has been created and maintained by Robin Gutell, University of Texas at Austin, and is publicly available on the Institute for Cellular and Molecular Biology web page on the world wide web of the Internet at, for example, “rna.icmb.utexas.edu/.” There is also a publicly available rRNA database created and maintained by the University of Antwerp, Belgium on the world wide web of the Internet at, for example, “rrna.uia.ac.be.”

These databases have been analyzed to determine regions that are useful as bioagent identifying amplicons. The characteristics of such regions include: a) between about 80 and 100%, or greater than about 95% identity among species of the particular bioagent of interest, of upstream and downstream nucleotide sequences which serve as sequence amplification primer sites; b) an intervening variable region which exhibits no greater than about 5% identity among species; and c) a separation of between about 30 and 1000 nucleotides, or no more than about 50-250 nucleotides, or no more than about 60-100 nucleotides, between the conserved regions.

As a non-limiting example, for identification of Bacillus species, the conserved sequence regions of the chosen bioagent identifying amplicon must be highly conserved among all Bacillus species while the variable region of the bioagent identifying amplicon is sufficiently variable such that the molecular masses of the amplification products of all species of Bacillus are distinguishable.

Bioagent identifying amplicons amenable to molecular mass determination are either of a length, size or mass compatible with the particular mode of molecular mass determination (e.g., ESI-MS) or compatible with a means of providing a predictable fragmentation pattern in order to obtain predictable fragments of a length compatible with the particular mode of molecular mass determination. Such means of providing a predictable fragmentation pattern of an amplification product include, but are not limited to, cleavage with restriction enzymes or cleavage primers, for example.

Identification of bioagents can be accomplished at different levels using intelligent primers suited to resolution of each individual level of identification. “Broad range survey” intelligent primers are designed with the objective of identifying a bioagent as a member of a particular division of bioagents. A “bioagent division” is defined as group of bioagents above the species level and includes but is not limited to: orders, families, classes, clades, genera or other such groupings of bioagents above the species level. As a non-limiting example, members of the Bacillus/Clostridia group or gamma-proteobacteria group may be identified as such by employing broad range survey intelligent primers such as primers that target 16S or 23S ribosomal RNA.

“Division-wide” intelligent primers are designed with an objective of identifying a bioagent at the species level. As a non-limiting example, a Bacillus anthracis, Bacillus cereus and Bacillus thuringiensis can be distinguished from each other using division-wide intelligent primers. Division-wide intelligent primers are not always required for identification at the species level because broad range survey intelligent primers may provide sufficient identification resolution to accomplishing this identification objective.

“Drill-down” intelligent primers are designed with an objective of identifying a sub-species characteristic of a bioagent. A “sub-species characteristic” is defined as a property imparted to a bioagent at the sub-species level of identification as a result of the presence or absence of a particular segment of nucleic acid. Such sub-species characteristics include, but are not limited to, strains, sub-types, pathogenicity markers such as antibiotic resistance genes, pathogenicity islands, toxin genes and virulence factors. Identification of such sub-species characteristics is often critical for determining proper clinical treatment of pathogen infections.

Although the use of PCR is suitable for embodiments of the present invention, other nucleic acid amplification techniques may also be used, including ligase chain reaction (LCR) and strand displacement amplification (SDA). The high-resolution MS technique allows separation of bioagent spectral lines from background spectral lines in highly cluttered environments. In some embodiments, amplicons are produced by RT-PCR, (rt) RT-PCR, qPCR, or similar techniques. In some embodiments, methods of the present invention are particularly useful for use with any amplification technique that: has the potential to produce an amplicon comprising five or more different nucleotides, has the potential to produce amplicons with non-templated adenylation, and/or benefits from reducing or eliminating the effects of carry-over contamination. In some embodiments, amplification systems which find use with the methods of this invention include the polymerase chain reaction system (U.S. Pat. Nos. 4,683,195; 4,683,202; and 4,965,188), the ligase amplification system (PCT Patent Publication No. 89/09835), the self-sustained sequence replication system (EP No. 329,822 and PCF Patent Publication No. 90/06995), the transcription-based amplification system (PCT Patent Publication No. 89/01050 and EP No. 310,229), and the Qβ RNA replicase system (U.S. Pat. No. 4,957,858). Each of the foregoing patents and publications is incorporated herein by reference.

In some embodiments, the present invention provides determining the mass and/or base composition of amplicons produced using a procedure to eliminate and/or reduce the effect of carryover contamination (See, e.g., U.S. Pat. No. 5,418,149; herein incorporated by reference in its entirety). In some embodiments, methods are provided for determining the mass and/or base composition of amplicons produced using a “sterilizing” method intended to prevent nucleic acids generated from a prior amplification reaction from serving as templates in a subsequent amplification reaction. In some embodiments, a sterilizing method comprises (a) mixing conventional (e.g., A, C, G) and unconventional nucleotides (e.g., U) into an amplification reaction system containing an amplification reaction mixture (e.g., primers containing A, C, G, and T, nucelobases) and a target nucleic acid sequence; (b) amplifying the target nucleic acid sequence to produce amplified products of nucleic acid having the unconventional nucleotides and conventional nucleotides incorporated therein; and (c) degrading any amplified product that contaminates a subsequent amplification mixture by hydrolyzing covalent bonds of the unconventional nucleotides. In some embodiments, amplicons produced using such an amplification sequence contain 5 or more different types of nucleotides (e.g., conventional (e.g., A, C, G, T)) and unconventional (e.g., U). In some embodiments, the present invention provides methods for determining the mass and or base composition of amplicons produced by such methods.

In some embodiments, the present invention provides mass spectrometry-based detection and identification (e.g., through base composition determination) of amplicons. Mass spectrometry (MS)-based detection of PCR products provides a means for determination of BCS that has several advantages. MS is intrinsically a parallel detection scheme without the need for radioactive or fluorescent labels, since every amplification product is identified by its molecular mass. Mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons. Intact molecular ions can be generated from amplification products using one of a variety of ionization techniques to convert the sample to gas phase. These ionization methods include, but are not limited to, electrospray ionization (ESI), matrix-assisted laser desorption ionization (MALDI) and fast atom bombardment (FAB). For example, MALDI of nucleic acids, along with examples of matrices for use in MALDI of nucleic acids, are described in WO 98/54751 (Genetrace, Inc.). Embodiments of the invention are described in connection with ESI-MS; however, this should not be viewed as limiting, and any suitable MS techniques find use with embodiments of the present invention. In some embodiments, masses and base compositions of amplicons are determined by ESI-MS.

In some embodiments, large DNAs and RNAs, or large amplification products therefrom, can be digested with restriction endonucleases prior to ionization. Thus, for example, an amplification product that was 10 kDa could be digested with a series of restriction endonucleases to produce a panel of, for example, 100 Da fragments. Restriction endonucleases and their sites of action are well known to the skilled artisan. In this manner, mass spectrometry can be performed for the purposes of restriction mapping.

Upon ionization, several peaks are observed from one sample due to the formation of ions with different charges. Averaging the multiple readings of molecular mass obtained from a single mass spectrum affords an estimate of molecular mass of the bioagent. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used in the methods of the present invention include, but are not limited to, Fourier transform ion cyclotron resonance mass spectrometry (FT-ICR-MS), ion trap, quadrupole, magnetic sector, time of flight (TOF), Q-TOF, and triple quadrupole.

In some embodiments, the present invention employs mass-modifying tags. For example, if a sample two or more targets of similar molecular mass, or if a single amplification reaction results in a product that has the same mass as two or more bioagent reference standards, they can be distinguished by using mass-modifying “tags.” In this embodiment of the invention, a nucleotide analog or “tag” is incorporated during amplification (e.g., a 5-(trifluoromethyl)deoxythymidine triphosphate) which has a different molecular weight than the unmodified base so as to improve distinction of masses. Such tags are described in, for example, PCT WO97/33000, which is incorporated herein by reference in its entirety. This further limits the number of possible base compositions consistent with any mass. For example, 5-(trifluoromethyl)deoxythymidine triphosphate can be used in place of dTTP in a separate nucleic acid amplification reaction. Measurement of the mass shift between a conventional amplification product and the tagged product is used to quantitate the number of thymidine nucleotides in each of the single strands. Because the strands are complementary, the number of adenosine nucleotides in each strand is also determined. In another amplification reaction, the number of G and C residues in each strand is determined using, for example, the cytidine analog 5-methylcytosine (5-meC) or propyne C. The combination of the A/T reaction and G/C reaction, followed by molecular weight determination, provides a unique base composition. Any suitable mass tags find use in embodiments of the present invention, and may be utilized for any useful purpose.

In some embodiments of the present invention, the mass modified nucleobase comprises one of the following: 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C.

In some embodiments, the present invention provides determining the mass and/or base composition of amplicons comprising one or more (e.g., 1, 2, 3, 4, 5, or more) different types of nucleotides (e.g., A, C, G, T, U). In some embodiments, methods are provided for determining the mass and/or base composition of amplicons comprising five or more (e.g., 5, 6, 7, 8, 9, 10, or more) different types of nucleotides. In some embodiments, methods are provide for determining the mass and/or base composition of amplicons comprising nucleotides comprising uridine, thymidine, adenosine, cytidine, guanosime, 4-acetylcytosine, 8-hydroxy-N6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, 2,6-diaminopurine, and other natural or non-natural nucleosides.

It is important to note that, in contrast to probe-based techniques, mass spectrometry determination of base composition does not require prior knowledge of the composition in order to make the measurement, only to interpret the results. In this regard, the present invention provides bioagent classifying information similar to DNA sequencing and phylogenetic analysis at a level sufficient to detect and identify a given bioagent. Furthermore, the process of determination of a previously unknown BCS for a given bioagent (for example, in a case where sequence information is unavailable) has downstream utility by providing additional bioagent indexing information with which to populate BCS databases. The process of future bioagent identification is thus greatly improved as more BCS indexes become available in the BCS databases.

The present methods allow extremely rapid and accurate detection and identification of amplicons and/or bioagents compared to existing methods. Furthermore, this rapid detection and identification is possible even when sample material is impure. The methods leverage ongoing biomedical research in virulence, pathogenicity, drug resistance and genome sequencing into a method which provides greatly improved sensitivity, specificity and reliability compared to existing methods, with lower rates of false positives. Thus, the methods are useful in a wide variety of fields and for a variety of applications, including, but not limited to, those discussed herein. In some embodiments, methods described herein find use in, for example: identification of infectious agents in biological samples, identifying an infectious agent that is potentially the cause of a health condition in a biological entity (e.g., a human, a mammal, a bird, a reptile, etc.), screening blood and other bodily fluids and tissues, detection of bioagents and/or biowarfare pathogens, detecting bioagents in organ donors and/or in organs from donors, pharmacogenetic analysis and medical diagnosis, detection and identification of blood-borne pathogens, emm-typing process to be carried out directly from throat swabs, serotyping of viruses, distinguishing between members of the Orthopoxvirus genus, distinguishing between viral agents of viral hemorrhagic fevers (VHF), diagnosis of a plurality of etiologic agents of a disease, detection and identification of pathogens in livestock, detecting the presence of antibiotic resistance and/or toxin genes in a bacterial species, etc.

In some embodiments, the present method can also be used to detect single nucleotide polymorphisms (SNPs), or multiple nucleotide polymorphisms, rapidly and accurately. A SNP is defined as a single base pair site in the genome that is different from one individual to another. The difference can be expressed either as a deletion, an insertion or a substitution, and is frequently linked to a disease state. Because they occur every 100-1000 base pairs, SNPs are the most frequently bound type of genetic marker in the human genome.

In some embodiments, the present invention also provides systems and kits for carrying out the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to fifty primer pairs, from one to twenty primer pairs, from one to ten primer pairs, or from two to five primer pairs.

In some embodiments, the kit comprises one or more broad range survey primer(s), division wide primer(s), or drill-down primer(s), or any combination thereof. If a given problem involves identification of a specific bioagent, the solution to the problem may require the selection of a particular combination of primers to provide the solution to the problem. A kit may be designed so as to comprise particular primer pairs for identification of a particular bioagent. In some embodiments, the primer pair components of any of these kits may be additionally combined to comprise additional combinations of broad range survey primers and division-wide primers so as to be able to identify a bacterium.

In some embodiments, the kit contains standardized calibration polynucleotides for use as internal amplification calibrants. Internal calibrants are described in commonly owned U.S. Pat. No. 7,956,175 which is incorporated herein by reference in its entirety.

In some embodiments, the kit comprises a sufficient quantity of reverse transcriptase (if RNA is to be analyzed for example), a DNA polymerase, uracil N-glycosylase (UNG), suitable nucleoside triphosphates (including alternative dNTPs such as inosine or modified dNTPs such as the 5-propynyl pyrimidines or any dNTP containing molecular mass-modifying tags such as those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. A kit may also comprise amplification reaction containers such as microcentrifuge tubes and the like. A kit may also comprise reagents or other materials for isolating bioagent nucleic acid or bioagent identifying amplicons from amplification, including, for example, detergents, solvents, or ion exchange resins which may be linked to magnetic beads. A kit may also comprise a table of measured or calculated molecular masses and/or base compositions of bioagents using the primer pairs of the kit.

Some embodiments of the kits are 96-well or 384-well plates with a plurality of wells containing any or all of the following components: dNTPs, buffer salts, Mg2+, betaine, and primer pairs. In some embodiments, a polymerase and/or uracil N-glycosylase (UNG) is also included in the plurality of wells of the 96-well or 384-well plates.

Some embodiments of the kit contain instructions for PCR and mass spectrometry analysis of amplification products obtained using the primer pairs of the kits.

In some embodiments, the present invention provides a database (e.g, as part of a kit or system) of base compositions of bioagent identifying amplicons defined by a given set of primer pairs. In some embodiments, the database is stored on a convenient computer readable medium such as a compact disk or USB drive, for example.

In some embodiments, a computer program stored on a computer formatted medium is provided (such as a compact disk or portable USB disk drive, for example). In some embodiments, programmed instructions which direct a processor to analyze data obtained from the use of the primer pairs of the present invention are provided. The instructions of the software transform data related to amplification products into a molecular mass or base composition which is a useful concrete and tangible result used in identification and/or classification of bioagents. In some embodiments, the kits of the present invention contain all of the reagents sufficient to carry out one or more of the methods described herein.

Embodiments, of the present invention provide and/or utilize the devices, compositions, systems, kits, and methods provided in U.S. Pat. Nos. 7,217,510, 7,226,739, 7,255,992, 7,666,588, 7,666,592, 7,714,275, 7,718,354, 7,741,036, 7,781,162, 7,956,175, and/or 7,964,343; and U.S. Pat App. Nos.: 20090047665, 20090148829, 20090148836, 20090148837, 20090182511, 20090220937, 20090311683, 20100070069, 20100075430, 20100128558, 20100129811, 20100136515, 20100184035, 20100190240, 20100204266, 20100219336, 20100240102, 20100291544, 20100317014, 20110028334, 20110045456, 20110065111, 20110091882, 20110097704, 20110105531, 20110118151, 20110143358, 20110151437, 20110 166040, 20110172925, and/or 20110177515, each of which is herein incorporated by reference in their entireties.

While the present invention has been described with specificity in accordance with certain of its embodiments, the following examples serve only to illustrate the invention and are not intended to limit the same.

### Experimental

### EXAMPLE 1

### Differentiation Between Isolate and CST Amplicons by ESI-MS

### Nucleic Acid Samples

DNA samples from Brucella melitensis Switzerland F6145 (Bm), Francisella tularensis Vienna (Ft), Ricinus communis Indian HC4 (Rc), Rickettsia prowazekii Breinl (Rp), Rickettsia rickettsii Bitterroot VR891 (Rr) and Ricketsia typhi Wilmington (Rt) were acquired from the National Bioforensic Repository Collection (Columbus, Ohio). Clostridium botulinum Type F 27321 (Cb) DNA were provided by Richard Robison (Brigham Young University, Provo, Utah). RNA from Nipah 199901924 Malaysia Prototype (Ni), Hendra Lung-1 strain (He) and Flexal BeAn 293022 (Fl) virus samples was extracted from cell lysates in Trizol LS (Invitrogen, Carlsbad, Calif.). Control synthetic templates (CSTs) were purchased from American International Biotechnology Services (AlBioTech, Glen Allen, Va.).

### (rt) RT-PCR

Serial dilutions of the isolate nucleic acids and CSTs were amplified by RT-PCR or reverse transcription RT-PCR using the AB17900 (Applied Biosystems, Foster City, Calif.). Five microliters of the DNA template and CSTs were amplified in replicates of six, using TaqMan™ 1000 RXN Gold with Buffer A (Applied Biosystems) into a final volume of 50 μl with 1× buffer. Ten replicates of no template control (NTC) were identical to the isolate and CST reactions, except for lacking a nucleic acid template. Reaction mixes contained dATP, dCTP. and dGTP each at 0.25 mM (Bm, Fl, Re) or 0.2 mM (Rp, Rt, Rr); dUTP at 0.5 mM (Bm, Ft, Rc) or 0.4 mM (Rp, Rt Rr); MgCl at 5 mM (Re, Rp, Rr, Rt), 6 mM (Ft), or 3.5 mM (Bm); forward and reverse primers each at 0.3 μM (Ft, Rc), 0.2 μM (Rp, Rt), 0.4 μM (Rr), or 0.6 μM forward and 0.3 μM reverse (Bm); probe at 0.2 μM (Rc, Rr), 0.3 μM (Rp, Rt), 0.4 μM (Ft), or 0.15 μM (Bm). Cycling conditions were 95° C. for 10 min followed by 45 cycles 95° C. for 15 s and 60° C. 1 min.

Five microliters of the RNA templates and CSTs were amplified in replicates of six, using SuperScriptIII Platinum One-Step Quantitative RT-PCR System w/ROX (Invitrogen) into a final reaction volume of 50 μl. Ten replicates of no template control (NTC) were identical to the isolate and CST reactions, except for lacking a nucleic acid template. Reaction mixes contained forward and reverse primers each at 0.9 μM (Ni), 0.3 μM (He), or 0.2 μM (Fl) and probes at 0.2 μM (Ni), 0.15 μM (He), or 0.1 μM (Fl); additional magnesium sulfate, was added at 2.5 mM to the He reaction only. Cycling conditions were 50° C. for 30 min, 95° C. for 10 min, followed by 45 cycles of 95° C. for 15 s and 60° C. for 1 min. Sequences for the primers (Eurofins MWG Operon, Huntsville, Ala.) and probes (Applied Biosystems or Integrated DNA Technologies, Coralville, Iowa) for the reactions are found in Table 1. Data was analyzed using the SDS software, version 2.3 (Applied Biosystems).

TABLE 1
Primer and probe sequences for Real-Time PCR

| Target | Reference |
|---|---|
| Brucella melitensis | NMRC^{a} |
| F-AGG TGG AAG GCA GCC TTC TG | |
| R-CGC TCC TAC GCC GGA T | |
| P-(FAM)AGC CTG ATA CTT CAG ACC ACC CCA GAC A (TAM) | |
| Francisella tularensis | NMRC^{a} |
| F-ATC CAA CAA TAA GTA TTA CTC TTG GTG TTC TA | |
| R-CAC TTG CTT GTA ATA TAC TCG AAA CTT TCT | |
| p-(FAM)CAA TTT TAG TTC TAA CAT TTG ATT TAA (MGB) | |
| Clostridium botulinum | (Fach, et al., 2009) |
| F-GCA ATA TAG GAT TAC TAG GTT TTC ATT C | |
| R-GAA ATA AAA CTC CAA AAG CAT CCA TT | |
| P-(FAM)TTG GTT GCT AGT AGT TGG TAT TAT AAC AA (BHQ) | |
| Ricinus communis | |
| F-CCT TAC AAG TGA TTC TAA TAT ACG GGA AAC | |
| R-CAT CAT TCT TGA ACA TCC ATC GTT | |
| P-(FAM)TGT CAA GAT CCT CTC TTG TGG CCC TGC (TAM) | |
| Rickettsia prowazekii | (Jiang, et al., 2003) |
| F-TCT TAA CAT AAC AGG GCA GGG TAT | |
| R-GCC CGC TAA GAT CAT TAG CGT | |
| P-(FAM)CCG AGC CAG CGC CAC CAT GCA CTT TTG TAA | |
| GAG GCT CGG (Dabcyl) | |
| Rickettsia rickettsii | (Jiang, et al., 2005) |
| F-ATA ACC CAA GAC TCA AAC TTT GGT A | |
| R-GCA GTG TTA CCG GGA TTG CT | |
| P-(FAM)CGC GAT CTT AAA GTT CCT AAT GCT ATA ACC | |
| CTT ACC GAT CGC G (BHQ) | |
| Rickettsia typhi | (Henry, et al., 2007) |
| F-TGG TAT TAC TGC TCA ACA AGC T | |
| R-CAG TAA AGT CTA TTG ATC CTA CAC C | |
| P-(FAM)CGC GAT CGT TAA TAG CAG CAC CAG CAT TAT | |
| CGC G (BHQ) | |
| Nipah virus | (Guillaume, et al., 2004) |
| F-CTG TTC TAT AGG TTC TTC CCC TTC AT | |
| R-GCA AGA GAG TAA TGT TCA GGC TAG AG | |
| P-(FAM)TGC AGG AGG TGT GCT CAT TGG AGG (TAM) | |
| Hendra virus | (Smith, et al., 2001) |
| F-CTT CGA CAA AGA CGG AAC CAA | |
| R-CCA GCT CGT CGG ACA AAA TT | |
| P-(FAM)TGG CAT CTT TCA TGC TCC ATC TCG G (TAM) | |
| Flexal Virus | |
| F-CGT GCC CTA AAC CAC ACA GA | |
| R-CCT TTC CTG ACC CAC CTG AC | |
| P-(FAM)TGC TCT TGT GGT TAT TAC AAC CTA CCA GGC | |
| AA (MGB) | |

For each target, forward (F), reverse (R) primers and probe (P) sequences are shown

### ESI-MS for Base Composition Analysis

To determine the precise molecular mass of both strands of the (rt) RT-PCR products, the samples were analyzed on ESI-MS (PLEX-ID (Abbott Laboratories, Carlsbad, Calif.)). The method for ESI-MS has been described using the first PCR/ESI-MS instrument, the Ibis T5000 biosensor (Sampath, et al., Emerg Inject Dis, 11, 373-379 2005; herein incorporated by reference in its entirety). Unambiguous base compositions (nA nG nC nT nU) were determined for both strands of the (rt) RT-PCR amplicons from their exact mass measurements.

### RT-PCR Product Sequencing

The Clostridium botulinum RT-PCR product was sequenced from the forward and reverse primers with BigDye Terminator 1.1 (Applied Biosystems) following the manufacturer's instructions on ABI Prism 3130 XL Genetic Analyzer (Applied Biosystems). Data from four forward and four reverse replicates were analyzed with Sequencher v 4.9 (Gene Codes Corp, Ann Arbor, Mich.).

### Differentiation Between Isolate and CST Amplicons by ESI-MS

Nucleic acids (DNA or RNA) from the organisms listed in Table 1, including viral, bacterial and plant species, and the associated CSTs from each were detected successfully by (rt) RT-PCR analysis. Serial dilutions of isolate and associated CST nucleic acids were analyzed by either (rt) RT-PCR (RNA isolates) or RT-PCR (DNA isolates). The products from the RNA templates contained 4 bases (A, G, C, T). The products from the DNA templates contained 5 bases (A, G, C, T, U), because the reaction conditions for the RT-PCR resulted in the incorporation of uracils. The DNA primers used in these reactions contained thymines. And the Taq polymerase incorporated uracils for the remainder of the product. Initial isolate nucleic acid concentrations were dependent on availability of template, and copy numbers were estimated from a standard curve derived from each associated CST. For each test nucleic acid and associated CST, replicate Ct values were determined. Reaction products were further analyzed by ESI-MS to determine precise base composition for differentiation between isolate and CST (rt) RT-PCR products.

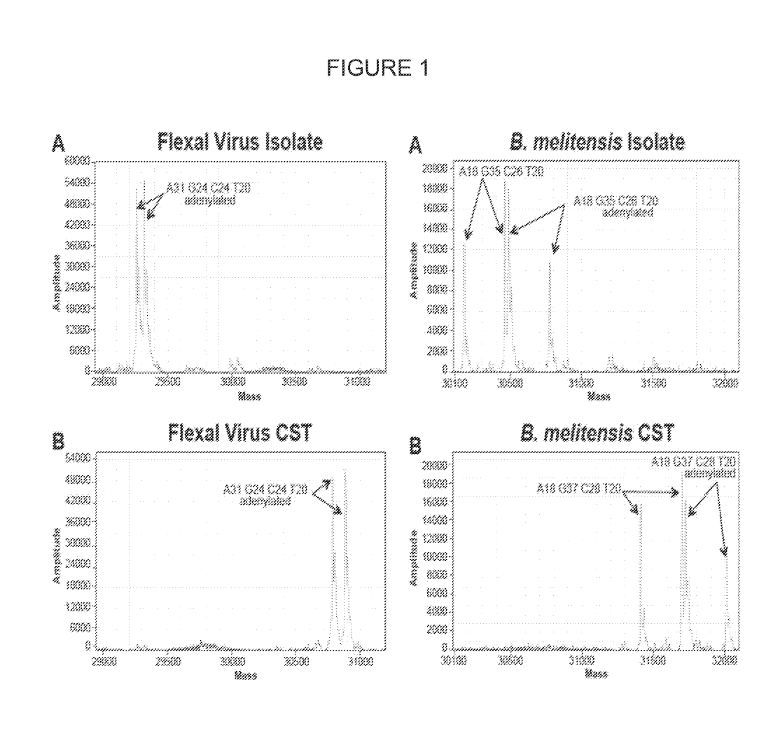

The contribution to the molecular mass of all five nucleotides is taken into account for RT-PCR products when determining the base composition of the forward and reverse strands using the ESI-MS method. Additionally, if the polymerase incorporates non-templated adenosines (Smith, et al. (1995) Genome Res, 5, 312-317.; herein incorporated by reference in its entirety), that factor must also be addressed during the calculations to determine base composition. The products of the RT-PCR and reverse transcription RT-PCR reactions comprised both nonadenylated and adenylated forms (SEE FIG. 1). Initial isolate nucleic acid concentrations were dependent on availability of template, and copy numbers were estimated from a standard curve derived from each associated CST. For each test isolate nucleic acid and associated CST, replicate Ct values were determined. Reaction products were further analyzed by ESI-MS to determine precise base composition for differentiation between isolate and CST RT-PCR or reverse transcription RT-PCR products.

Average Ct values and the number of positive replicates for the templates are listed in Table 2 (D A isolates) and Table 3 (RNA isolates). Sensitivity of (rt) RT-PCR for target nucleic acids at the lowest dilution ranged from single to lens of copies, depending on the isolate. Because contamination carryover is an important issue with highly sensitive assays it was important to differentiate between isolate and CST RT-PCR products. However, differentiation between isolate and CST products was not possible by (rt) RT-PCR analysis. Therefore the samples were subjected to ESI-MS base composition analysis to specifically identify the products in each reaction.

TABLE 2
Correlation of isolate and CST RT-PCR and ESI-MS positive identifications

| | | RT-PCR | | ESI-MS | Forward Strand |
|---|---|---|---|---|---|
| Target | Copies^{a} | Positives | Av Ct^{b} | Positives^{c} | Mass Spec ID^{d} |
| B. melitensis | 800 | 6/6 | 30.43 | 6/6 | A18 G35 C26 T20 |
| isolate | 40 | 6/6 | 34.63 | 6/6 | A18 G35 C26 T20 |
| | 4 | 6/6 | 38.17 | 6/6 | A18 G35 C26 T20 |
| | 0.4 | 2/6 | 40.25 | 2/6 | A18 G35 C26 T20 |
| CST | 100,000 | 6/6 | 23.25 | 6/6 | A18 G37 C28 T20 |
| | 10,000 | 6/6 | 26.46 | 6/6 | A18 G37 C28 T20 |
| | 1000 | 6/6 | 29.84 | 6/6 | A18 G37 C28 T20 |
| | 100 | 6/6 | 33.28 | 6/6 | A18 G37 C28 T20 |
| | 10 | 6/6 | 36.68 | 6/6 | A18 G37 C28 T20 |
| NTC | 0 | 0/10 | NA | 0/10 | NA |
| F. tularensis | 60,000 | 6/6 | 24.41 | 6/6 | A38 G15 C11 T29 |
| isolate | 6800 | 6/6 | 27.74 | 6/6 | A38 G15 C11 T29 |
| | 700 | 6/6 | 31.41 | 6/6 | A38 G15 C11 T29 |
| | 90 | 6/6 | 34.60 | 6/6 | A38 G15 C11 T29 |
| | 10 | 6/6 | 38.4 | 6/6 | A38 G15 C11 T29 |
| CST | 100,000 | 6/6 | 23.58 | 6/6 | A38 G17 C14 T29 |
| | 10,000 | 6/6 | 26.95 | 6/6 | A38 G17 C14 T29 |
| | 1000 | 6/6 | 30.83 | 6/6 | A38 G17 C14 T29 |
| | 100 | 6/6 | 34.17 | 6/6 | A38 G17 C14 T29 |
| | 10 | 6/6 | 38.05 | 6/6 | A38 G17 C14 T29 |
| NTC | 0 | 0/10 | NA | 0/10 | NA |
| R. communis | 90 | 6/6 | 32.58 | 6/6 | A25 G21 C22 T27 |
| isolate | 10 | 6/6 | 35.97 | 6/6 | A25 G21 C22 T27 |
| | 1 | 4/6 | 39.12 | 6/6 | A25 G21 C22 T27 |
| CST | 100,000 | 6/6 | 22.33 | 6/6 | A25 G24 C23 T27 |
| | 10,000 | 6/6 | 25.66 | 6/6 | A25 G24 C23 T27 |
| | 1000 | 6/6 | 29.00 | 6/6 | A25 G24 C23 T27 |
| | 100 | 6/6 | 32.23 | 6/6 | A25 G24 C23 T27 |
| | 10 | 6/6 | 35.97 | 6/6 | A25 G24 C23 T27 |
| NTC | 0 | 0/10 | NA | 0/10 | NA |
| R. prowazekii | 200,000 | 6/6 | 25.48 | 6/6 | A33 G23 C25 T38 |
| isolate | 20,000 | 6/6 | 28.82 | 6/6 | A33 G23 C25 T38 |
| | 2000 | 6/6 | 32.23 | 6/6 | A33 G23 C25 T38 |
| | 200 | 6/6 | 35.68 | 6/6 | A33 G23 C25 T38 |
| | 20 | 6/6 | 39.47 | 6/6 | A33 G23 C25 T38 |
| CST | 100,000 | 6/6 | 26.49 | 6/6 | A33 G26 C28 T38 |
| | 10,000 | 6/6 | 29.9 | 6/6 | A33 G26 C28 T38 |
| | 1000 | 6/6 | 33.29 | 6/6 | A33 G26 C28 T38 |
| | 100 | 6/6 | 36.71 | 6/6 | A33 G26 C28 T38 |
| | 10 | 6/6 | 40.41 | 6/6 | A33 G26 C28 T38 |
| NTC | 0 | 0/10 | NA | 0/10 | NA |
| R. rickettsii | 200,000 | 6/6 | 25.61 | 6/6 | A37 G28 C21 T39 |
| isolate | 20,000 | 6/6 | 28.89 | 6/6 | A37 G28 C21 T39 |
| | 2000 | 6/6 | 32.31 | 6/6 | A37 G28 C21 T39 |
| | 200 | 6/6 | 35.17 | 6/6 | A37 G28 C21 T39 |
| | 20 | 6/6 | 39.53 | 6/6 | A37 G28 C21 T39 |
| CST | 100,000 | 6/6 | 26.61 | 6/6 | A37 G31 C24 T39 |
| | 10,000 | 6/6 | 29.82 | 6/6 | A37 G31 C24 T39 |
| | 1000 | 6/6 | 33.17 | 6/6 | A37 G31 C24 T39 |
| | 100 | 6/6 | 36.71 | 6/6 | A37 G31 C24 T39 |
| | 10 | 6/6 | 40.84 | 6/6 | A37 G31 C24 T39 |
| NTC | 0 | 0/10 | NA | 0/10 | NA |
| R. typhi | 210,000 | 6/6 | 22.96 | 6/6 | A39 G20 C25 T38 |
| isolate | 22,000 | 6/6 | 26.30 | 6/6 | A39 G20 C25 T38 |
| | 2200 | 6/6 | 29.63 | 6/6 | A39 G20 C25 T38 |
| | 220 | 6/6 | 33.03 | 6/6 | A39 G20 C25 T38 |
| | 20 | 6/6 | 36.58 | 6/6 | A39 G20 C25 T38 |
| CST | 100,000 | 6/6 | 24.09 | 6/6 | A39 G23 C28 T38 |
| | 10,000 | 6/6 | 27.39 | 6/6 | A39 G23 C28 T38 |
| | 1000 | 6/6 | 30.74 | 6/6 | A39 G23 C28 T38 |
| | 100 | 6/6 | 34.30 | 6/6 | A39 G23 C28 T38 |
| | 10 | 6/6 | 37.57 | 6/6 | A39 G23 C28 T38 |
| NTC | 0 | 0/10 | NA | 0/10 | NA |

CST = control synthetic template
NTC = no template control
NA—Not applicable
^{a}For isolate, copy numbers are estimated from standard curve derived from CST.
^{b}Average Ct values reflect the number of positives (out of 6) as reported in the RT-PCR Positive column
^{c}ESI-MS positives reflect the number of samples that produced clearly defined peaks on the mass spectra of the correct MW for both the forward and reverse strands with and/or without adenylation.
^{d}Reported values are for the native strand (non-adenylated).

TABLE 3
Correlation of isolate and CST rt RT-PCR and ESI-MS positive identifications

| | | rt RT-PCR | | ESI-MS | Forward Strand |
|---|---|---|---|---|---|
| Target | Copies^{a} | Positives | Av Ct^{b} | Positives^{c} | Mass Spec ID^{d} |
| Nipah Virus | 1.5 × 10⁷ | 6/6 | 16.13 | 6/6 | A36 G35 C14 T21 |
| isolate | 1.5 × 10⁶ | 6/6 | 19.75 | 6/6 | A36 G35 C14 T21 |
| | 200,000 | 6/6 | 24.04 | 6/6 | A36 G35 C14 T21 |
| | 16,000 | 6/6 | 27.69 | 6/6 | A36 G35 C14 T21 |
| | 1400 | 6/6 | 31.03 | 6/6 | A36 G35 C14 T21 |
| | 120 | 6/6 | 34.49 | 6/6 | A36 G35 C14 T21 |
| CST | 100,000 | 6/6 | 25.07 | 6/6 | A35 G36 C16 T21 |
| | 10,000 | 6/6 | 28.41 | 6/6 | A35 G36 C16 T21 |
| | 1000 | 6/6 | 31.54 | 6/6 | A35 G36 C16 T21 |
| | 100 | 6/6 | 34.57 | 6/6 | A35 G36 C16 T21 |
| | 10 | 1/6 | 37.86 | 4/6 | A35 G36 C16 T21 |
| NTC | 0 | 0/10 | NA | 0/10 | NA |
| Hendra Virus | 3000 | 6/6 | 28.43 | 6/6 | A17 G17 C18 T17 |
| isolate | 300 | 6/6 | 31.90 | 6/6 | A17 G17 C18 T17 |
| | 30 | 6/6 | 35.3 | 6/6 | A17 G17 C18 T17 |
| | 5 | 6/6 | 37.88 | 6/6 | A17 G17 C18 T17 |
| CST | 100,000 | 6/6 | 23.47 | 6/6 | A17 G19 C21 T17 |
| | 10,000 | 6/6 | 26.76 | 6/6 | A17 G19 C21 T17 |
| | 1000 | 6/6 | 30.18 | 6/6 | A17 G19 C21 T17 |
| | 100 | 6/6 | 33.61 | 6/6 | A17 G19 C21 T17 |
| | 10 | 6/6 | 36.84 | 6/6 | A17 G19 C21 T17 |
| NTC | 0 | 0/10 | NA | 0/10 | NA |
| Flexal Virus | 100,000 | 6/6 | 21.56 | 6/6 | A31 G21 C22 T20 |
| isolate | 10,000 | 6/6 | 24.95 | 6/6 | A31 G21 C22 T20 |
| | 1,000 | 6/6 | 28.33 | 6/6 | A31 G21 C22 T20 |
| | 95 | 6/6 | 31.80 | 6/6 | A31 G21 C22 T20 |
| | 11 | 6/6 | 35.02 | 6/6 | A31 G21 C22 T20 |
| | 1 | 6/6 | 38.28 | 6/6 | A31 G21 C22 T20 |
| CST | 100,000 | 6/6 | 21.60 | 6/6 | A31 G24 C24 T20 |
| | 10,000 | 6/6 | 24.96 | 6/6 | A31 G24 C24 T20 |
| | 1000 | 6/6 | 28.34 | 6/6 | A31 G24 C24 T20 |
| | 100 | 6/6 | 31.62 | 6/6 | A31 G24 C24 T20 |
| | 10 | 6/6 | 35.19 | 6/6 | A31 G24 C24 T20 |
| NTC | 0 | 0/10 | NA | 0/10 | NA |

CST = control synthetic template
NTC = no template control
NA—not applicable
^{a}For isolate, copy numbers are estimated from standard curve derived from CST.
^{b}Avetage Ct values reflect the number of positives (out of 6) as reported in the rt RT-PCR Positive column
^{c}ESI-MS positives reflect the number of samples that produced clearly defined peaks on the mass spectra of the correct MW for both the forward and reverse strands with and/or without adenlyation
^{d}Reported values are for the native strand (non-adenylated).

ESI-MS was used to analyze the (rt) RT-PCR amplicons to identify the specific products as described (Chen, et al., Diagn Microbiol Infect Dis, 69, 179-186 2011). Representative mass spectra for an (rt) RT-PCR (Flexal virus) reaction and a RT-PCR reaction (B. melitensis) are provided (SEE FIG. 1). The shift in molecular weight between the isolate and CST was clearly discernible. The products from the Flexal virus reaction were determined to be non-template fully adenylated as determined by the ESI-MS. However, the products from the B. melitensis reaction contained both non-templated adenylated strands and non-adenylated strands. A variety of adenylated and non-adenylated product patterns were observed from the isolates and CSTs analyzed. The base compositions for the forward strand of each isolate and CST are reported in Tables 2 and 3. Table 2 base counts were calculated taking into account the presence of both uracils and thymines but for clarity, were reported as if thymines only were incorporated. Likewise, throughout, the reported base counts do not reflect any non-templated adenylations.

Identical base compositions were determined at each dilution for all targets and were reflected by the expected differences between isolate and associated CST (rt) RT-PCR products. All (rt) RT-PCR positive reactions were detected by ESI-MS, however there were instances of ESI-MS detection of amplicons that did not result in defined Ct values from the (rt) RT-PCR reactions (Table 2, R. communis isolate copy level of 1, 4/6 PCR positives vs. 6/6 MS positives; Table 3, Nipah virus CST copy level of 10, 1/6 PCR positive vs. 4/6 MS positives).

### SNP Detection and Verification

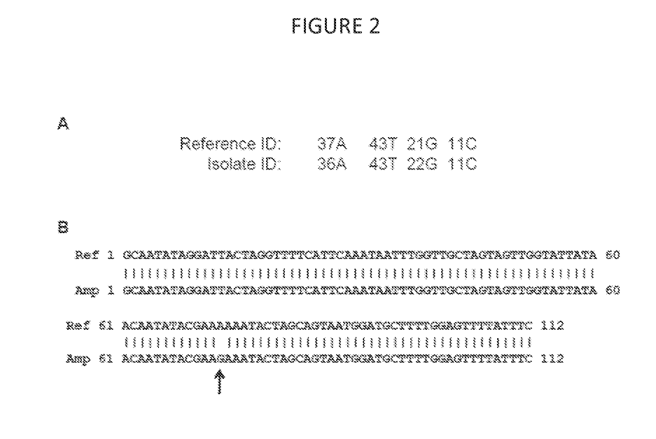

The sensitivity of the ESI-MS allowed detection of an SNP between the C. botulinum F RT-PCR product and the composition reported for the reference in GenBank (Accession CP000728.1). The detected base count from the isolate nucleic acid differed from the predicted reference GenBank base count by an A-G SNP (SEE FIG. 2A). Subsequent sequence analysis of the purified isolate amplicon DNA confirmed the A-G SNP transition (SEE FIG. 2B).

### EXAMPLE 2

### Amplicon Sizing

The National Bioforensics Analysis Center (NBFAC) implements processes designed to control and identify signature cross-contamination to ensure that results generated from analyses of evidentiary material are unimpeachable. One of the methods currently utilized by NBFAC in real-time PCR assays is the application of mutagenized positive control templates to ensure that amplicons generated from positive control templates can be distinguished from amplicons generated from wild type sequence. The mutagenized templates (MT) contain an insertion which is located within the predicted amplicon, but not within either the primer or probe binding sequences. All amplicons generated are less than 150 base pairs. NBFAC currently sequences the amplification products to distinguish wild type amplicons from mutagenized template amplicons. However, this process is time consuming and it is not amenable to high throughput analysis. Experiments were conducted during development of embodiments of the present invention to demonstrate the capability of the methods described herein to meet the requirements of the NBFAC for distinguishing control and unknown amplicons generated in real-time PCR assays. Molecular mass and base composition analysis of RT-PCR amplicons were performed by electrospray ionization-mass spectrometry on the IBIS BIOSCIENCES T5000 platform.

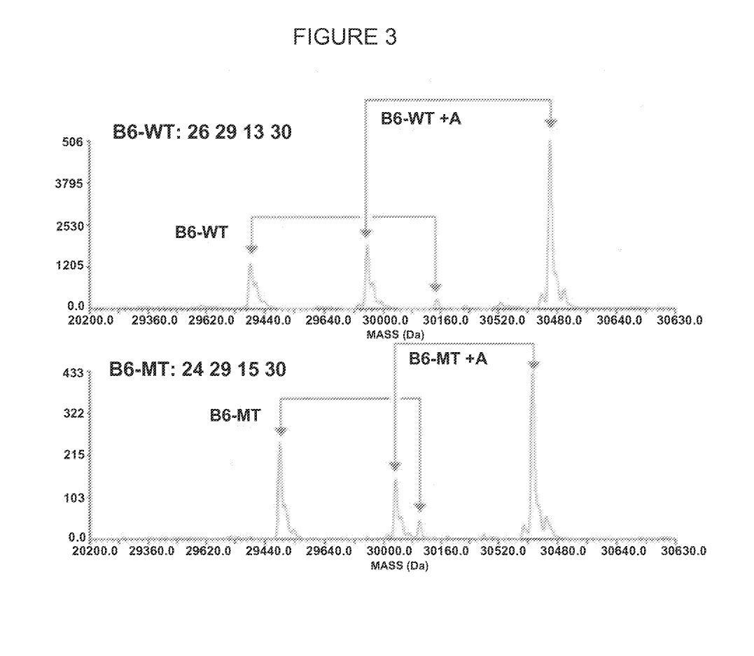

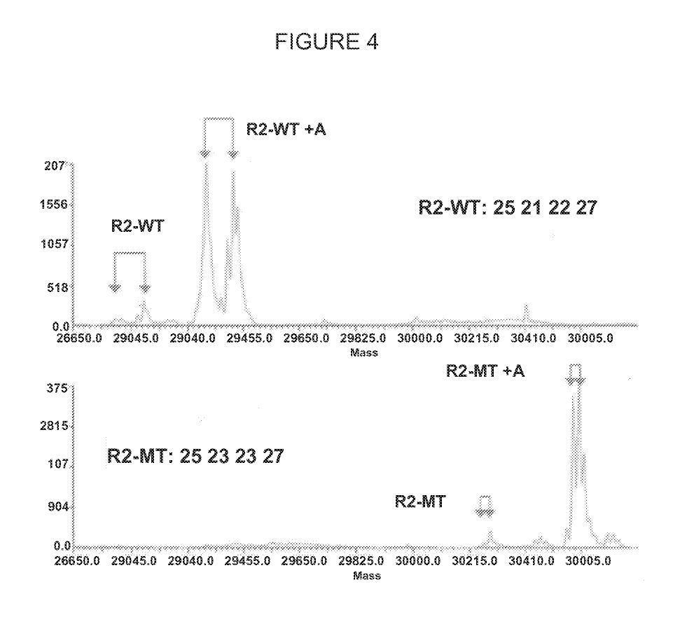

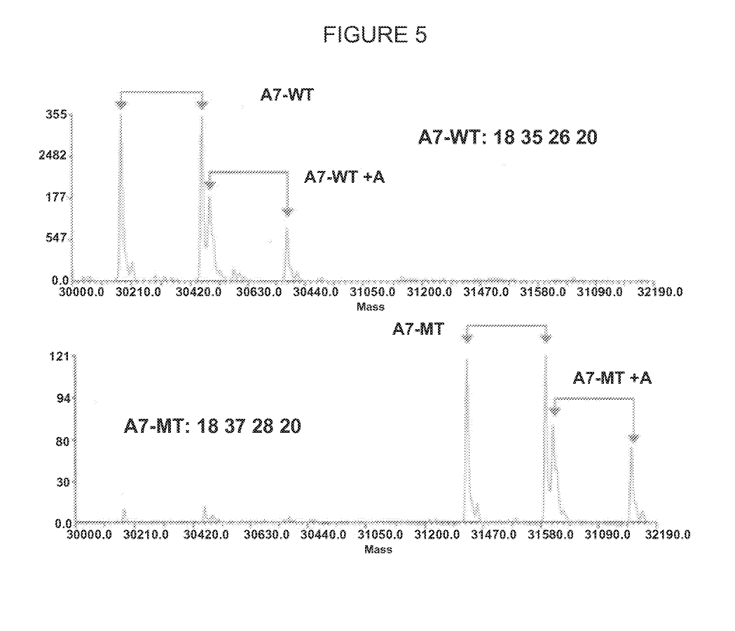

Three sets of NBFAC samples analyzed, as described below. The first set of samples consisted of the unblinded WT and MT PCR amplicons from A7, B6, L4, and R2 assays and their corresponding amplicon and PCR primer sequences. Samples were analyzed using the Ibis T5000 system. Following de-salting and processing on the T500 system, the A7, B6, and R2 amplicons, both forward and reverse strands were identified for both the WT and MT amplicons (SEE FIGS. 3-5). As expected from the PCR conditions used to generate these amplicons, there was a high level of adenylated amplicon; this adenylation is due to a property of Taq polymerase to add a non-templated adeninine to the PCR amplicons and does not affect the ability to resolve the MT and WT amplicons. To confirm the presence of the amplicons and estimate their levels they were also analyzed with the Agilent Bioanalyzer. The masses and base compositions for these amplicons were determined using the T5000. One of the amplicons, (R2-MT), was found to have an additional G in the amplicon that was in contrast to the amplicon sequence provided. This discrepancy was found to be a typo in the sequence provided and the base composition signature identified using the T5000 was confirmed to be correct.

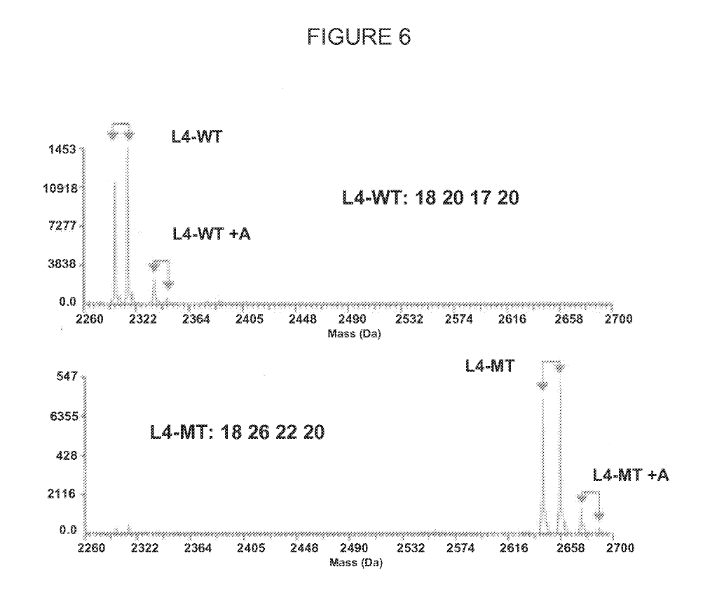

Analysis of the L4 amplicons required re-PCR of the amplicon as the amplicon appeared to be heterogeneous and at low levels, based upon ESI-MS and by analysis using the Agilent Bioanalyzer. This was also found to be true for a second aliquot of L4 amplicons. The L4 amplicons were generated using a proprietary ABI PCR mastermix. This PCR Mastermix was only for the L4 reactions and was not used to generate the other amplicons. Upon re-PCR the L4 amplicons were readily resolvable (SEE FIG. 6). The NBFAC-provided L4-WT and L4-MT amplicons were used as template in a second PCR amplification under standard Ibis PCR conditions. Specifically the PCR was performed in a 40 μl reaction containing 3 U of AmpliTaq Gold (Applied Biosystems, Foster City, Calif.), 20 mM Tris (pH 8.3), 75 mM KCl, 1.5 mM MgCl₂, 20 mM sorbitol (Sigma Corp, St Louis, Mo.), 0.4 M betaine (Sigma Corp, St Louis, Mo.), 800 μM equal mix of dCTP, dTTP, dGTP, and dATP, and 250 nM of each primer. The following PCR cycling conditions were used: 95° C. for 10 min, followed by 8 cycles of 95° C. for 30 s, 48° C. for 30 s, and 72° C. for 30 s, with the 48° C. annealing temperature increasing 0.9° C. each cycle. The PCR was then continued for 37 additional cycles of 95° C. for 15 s, 56° C. for 20 s, and 72° C. for 20 s. The PCR cycle ended with a final extension of 2 min at 72° C. followed by a 4° C. hold. The L4 PCR primers were modified to have a T at their 5 prime ends to suppress the adenylation of the PCR amplicons.

Eight blinded amplicon samples were obtained from NBFAC and were directly analyzed by ESI-MS, and in parallel each sample was amplified in a secondary PCR with the L4 primers. Observed basecounts for the nonadenylated and adenylated products were matched to the expected basecounts of the WT and MT amplicons for each assay. (Table 4). Samples 7 and 8 required re-PCR with the L4 primers and the re-PCR amplicons matched the L4-WT and L4-MT expected amplicons in Table 1. All T5000 reported amplicons matched the expected amplicons.

TABLE 4
Results of Analysis of Blinded Samples

| NBFAC | T5000 | |
|---|---|---|
| Sample ID | Identification | Expected ID |
| Sample 1 | B6-WT | B6-WT |
| Sample 2 | B6-MT | B6-MT |
| Sample 3 | R2-WT | R2-WT |
| Sample 4 | R2-MT | R2-MT |
| Sample 5 | A7-WT | A7-WT |
| Sample 6 | A7-MT | A7-MT |
| Sample 7 | L4-WT | L4-WT |
| Sample 8 | L4-MT | L4-MT |

### EXAMPLE 3

### Differentiating Microbial Forensic Real-Time PCR Target and Control Products by Electrospray Ionization Mass Spectrometry

### Materials and Methods

### Isolate and Control Nucleic Acid Samples

DNA samples from Brucella melitensis Switzerland F6145 (Bm), Francisella tularensis Vienna (Ft), Ricinus communis Indian HC4 (Rc), Rickettsia prowazekii Breinl (Rp), Rickettsia rickettsii Bitterroot VR891 (Rr) and Rickettsia typhi Wilmington (Rt) were acquired from the National Bioforensic Repository Collection (Columbus, Ohio). Clostridium botulinum Type F 27321 (Cb) DNA was provided by Richard Robison (Brigham Young University, Provo, Utah). RNA from Nipah 199901924 Malaysia Prototype (Ni), Hendra Lung-1 strain (He), and Flexal BeAn 293022 (Fl) virus samples was extracted from cell lysates in Trizol LS (Invitrogen, Carlsbad, Calif.). Control synthetic templates (CSTs) were purchased from American International Biotechnology Services (AlBioTech, Glen Allen, Va.).

### RT-PCR and Reverse Transcriptase RT-PCR Reactions

Serial dilutions of the nucleic acids (CSTs and isolate) were amplified by RT-PCR or reverse transcriptase RT-PCR using the AB17900 (Applied Biosystems, Foster City, Calif.). Data was analyzed using the SDS software, version 2.3 (Applied Biosystems). DNA templates and CSTs (5 μl) were amplified in replicates of six, using TaqMan™ 1000 RXN Gold with Buffer A (Applied Biosystems) in a final volume of 50 μl in 1× buffer. Ten replicates of no template control (NTC) were identical to the isolate and CST reactions, but lacked a nucleic acid template. Reaction mixes contained dATP, dCTP, and dGTP each at 0.25 mM (Bm, Ft, Rc) or 0.2 mM (Rp, Rt, Rr); dUTP at 0.5 mM (Bm, Ft, Rc) or 0.4 mM (Rp, Rt, Rr); MgCl₂ at 5 mM (Rc, Rp, Rr, Rt), 6 mM (Ft), or 3.5 mM (Bm); forward and reverse primers each at 0.3 μM (Ft, Rc), 0.2 μM (Rp, Rt), 0.4 μM (Rr), or 0.6 μM forward and 0.3 μM reverse (Bm); probe at 0.2 μM (Rc, Rr), 0.3 μM (Rp, Rt), 0.4 μM (Ft), or 0.15 μM (Bm). Cycling conditions were 95° C. for 10 min followed by 45 cycles of 95° C. for 15 s and 60° C. 1 min. Sequences for the primers (Eurofins MWG Operon, Huntsville, Ala.) and probes (Applied Biosystems or Integrated DNA Technologies, Coralville, Iowa) for the reactions can be found in Table 5 (Fach et al., J Appl Microbiol 2009; 107(2):465-73; Henry et al., Mol Cell Probes 2007; 21(1):17-23; Jiang et al., Int Rev Armed Forces Med Serv 2005; 78:174-9; Jiang et al., Ann N Y Acad Sci 2003; 990:302-10).

RNA templates and CSTs (5 μl) were amplified in replicates of six using SuperScriptIII Platinum One-Step Quantitative RT-PCR System w/ROX (Invitrogen) in a final reaction volume of 50 μl. Ten replicates of NTC were identical to the isolate and CST reactions, but lacked a nucleic acid template. Reaction mixes contained forward and reverse primers each at 0.9 μM (Ni), 0.3 μM (He), or 0.2 μM (Fl) and probes at 0.2 μM (Ni), 0.15 μM (He), or 0.1 μM (Fl); additional MgSO₄ was added at 2.5 mM to the He reaction only. Cycling conditions were 50° C. for 30 min, 95° C. for 10 min, followed by 45 cycles of 95° C. for 15 s and 60° C. for 1 min. Sequences for the primers (Eurofins MWG Operon) and probes (Applied Biosystems or Integrated DNA Technologies) for the reactions can be found in Table 5 (Guillaume et al., J Virol Methods 2004; 120(2):229-37; Smith et al., J Virol Methods 2001; 98(1):33-40).

### Mass Spectrometry for Base Composition Analysis

To determine the precise molecular mass of both strands of the RT-PCR and reverse transcriptase RT-PCR products, the samples were analyzed by ESI-MS on a PLEX-ID (Abbott Laboratories, Carlsbad, Calif.). The method used was essentially that described using the PCR/ESI-MS instrument, Ibis T5000 biosensor (Sampath et al., Emerg Infect Dis 2005; 11(3):373-9). Unambiguous base compositions (nA nG nC nT nU) were determined for both strands of the RT-PCR and reverse transcriptase RT-PCR amplicons from their exact mass measurements.

### RT-PCR Product Sequencing

The Clostridium botulinum RT-PCR product was sequenced from the forward and reverse primers with BigDye Terminator 1.1 (Applied Biosystems) following the manufacturer's instructions on ABI Prism 3130 XL Genetic Analyzer (Applied Biosystems). Data from four forward and four reverse replicates were analyzed with Sequencher v 4.9 (Gene Codes Corp, Ann Arbor, Mich.).

### Template Mixture Experiment

Rt-PCR and rt RT-PCR reactions were performed as described except that CST and isolates were intentionally mixed prior to amplification to mimic a contamination event. Rickettsia rickettsii isolate and CST were combined at approximately 10,000 isolate and 100 CST copies as an example for RT-PCR. Flexal virus isolate and CST were combined at approximately 100 isolate and 10 CST copies as an example for reverse transcriptase RT-PCR.

TABLE 5
Target
Brucella melitensis
F-AGG TGG AAG GCA GCC TTC TG
R-CGC TCC TAC GCC GGA T
P-(FAM)AGC CTG ATA CTT CAG ACC ACC CCA GAC A (TAM)
Francisella tularensis
F-ATC CAA CAA TAA GTA TTA CTC TTG GTG TTC TA
R-CAC TTG CTT GTA ATA TAC TCG AAA CTT TCT
p-(FAM)CAA TTT TAG TTC TAA CAT TTG ATT TAA (MGB)
Clostridium botulinum
F-GCA ATA TAG GAT TAC TAG GTT TTC ATT C
R-GAA ATA AAA CTC CAA AAG CAT CCA TT
P-(FAM)TTG GTT GCT AGT AGT TGG TAT TAT AAC AA (BHQ)
Ricinus communis
F-CCT TAC AAG TGA TTC TAA TAT ACG GGA AAC
R-CAT CAT TCT TGA ACA TCC ATC GTT
P-(FAM)TGT CAA GAT CCT CTC TTG TGG CCC TGC (TAM)
Rickettsia prowazekii
F-TCT TAA CAT AAC AGG GCA GGG TAT
R-GCC CGC TAA GAT CAT TAG CGT
P-(FAM)CCG AGC CAG CGC CAC CAT GCA CTT TTG TAA
GAG GCT CGG (Dabcyl)
Rickettsia rickettsii
F-ATA ACC CAA GAC TCA AAC TTT GGT A
R-GCA GTG TTA CCG GGA TTG CT
P-(FAM)CGC GAT CTT AAA GTT CCT AAT GCT ATA ACC
CTT ACC GAT CGC G (BHQ)
Rickettsia typhi
F-TGG TAT TAC TGC TCA ACA AGC T
R-CAG TAA AGT CTA TTG ATC CTA CAC C
P-(FAM)CGC GAT CGT TAA TAG CAG CAC CAG CAT TAT
CGC G (BHQ)
Nipah virus
F-CTG TTC TAT AGG TTC TTC CCC TTC AT
R-GCA AGA GAG TAA TGT TCA GGC TAG AG
P-(FAM)TGC AGG AGG TGT GCT CAT TGG AGG (TAM)
Hendra virus
F-CTT CGA CAA AGA CGG AAC CAA
R-CCA GCT CGT CGG ACA AAA TT
P-(FAM)TGG CAT CTT TCA TGC TCC ATC TCG G (TAM)
Flexal Virus
F-CGT GCC CTA AAC CAC ACA GA
R-CCT TTC CTG ACC CAC CTG AC
P-(FAM)TGC TCT TGT GGT TAT TAC AAC CTA CCA GGC
AA (MGB)

### Results

### Differentiation Between Isolate and Positive Control Synthetic Template Amplicons by ESI-MS

Nucleic acids (DNA or RNA) from the organisms listed in Table 5, including viral, bacterial and plant species, and the associated CSTs from each were detected successfully by RT-PCR and reverse transcriptase RT-PCR analysis. Serial dilutions (six replicates each) of isolate and associated CST nucleic acids were analyzed by either RT-PCR (DNA isolates) or reverse transcriptase RT-PCR (RNA isolates). The reverse transcriptase RT-PCR reaction products from the RNA templates contained four bases (A, G, C, and T). The RT-PCR products from the DNA templates contained five bases (A, G, C, T, and U), because the reaction conditions for the RT-PCR resulted in the incorporation of uracils. The DNA primers used in these reactions contained thiamines, while the Taq™ polymerase incorporated uracils for the remainder of the product.

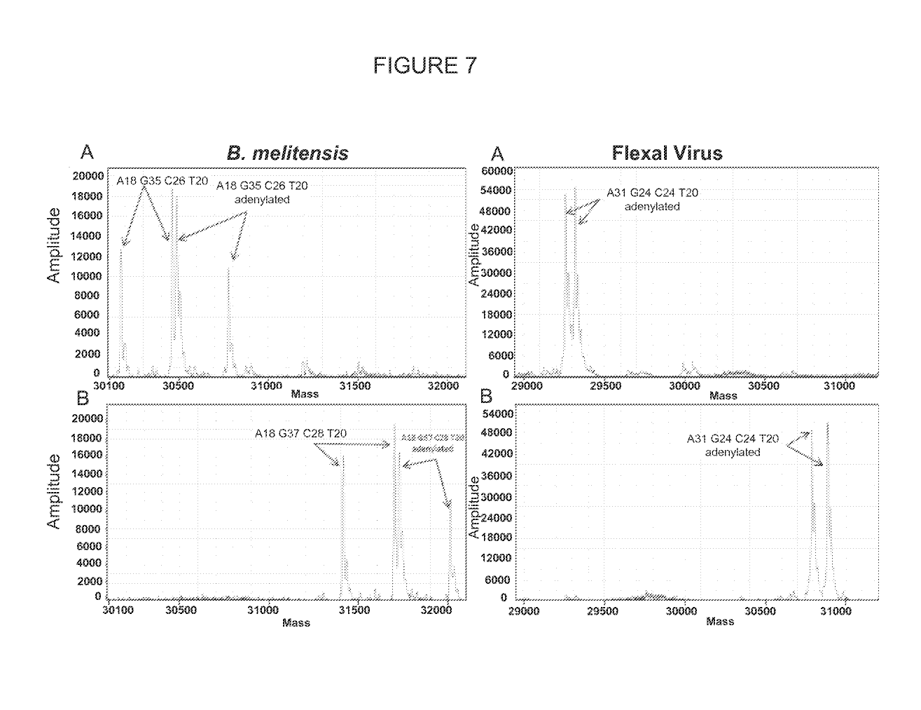

The contribution to the molecular mass of all five nucleotides is taken into account for RT-PCR products when determining the base composition of the forward and reverse strands using the ESI-MS method. Additionally, if the polymerase incorporates non-templated adenosines (Smith et al., Genome Res 1995; 5(3):312-7), that factor is also addressed during the calculations to determine base composition. The products of the RT-PCR and reverse transcriptase RT-PCR reactions comprised both non-adenylated and adenylated forms (see FIG. 7).

Initial test isolate nucleic acid concentrations were dependent on availability of template, and copy numbers were estimated from a standard curve derived from each associated CST. For each test isolate nucleic acid and associated CST, replicate Ct values were determined. Reaction products were further analyzed by ESI-MS to determine precise base composition for differentiation between isolate and CST RT-PCR or reverse transcriptase RT-PCR products. The forward strand base compositions determined for the amplicon products are shown in Table 6.

Average Ct values and the number of positive replicates for the templates are listed in Table 7 (DNA isolates) and Table 8 (RNA isolates). Sensitivity of RT-PCR and reverse transcriptase RT-PCR for target nucleic acids at the lowest dilution ranged from a single copy to tens of copies, depending on the isolate. Because contamination carryover is an important issue with highly sensitive assays, it was important to differentiate between isolate and CST products. However, differentiation between isolate and CST products was not possible by RT-PCR or reverse transcriptase RT-PCR analysis. Therefore the samples were subjected to ESI-MS base composition analysis to specifically identify the products in each reaction as described (Chen et al., Diagn Microbiol Infect Dis 2011; 69(2):179-86).

Representative mass spectra for an RT-PCR reaction (B. melitensis) and a reverse transcriptase RT-PCR (Flexal virus) reaction are shown in FIG. 7. The shift in molecular weight between the isolate and CST was clearly discernible. All detected products from the Flexal virus reaction contained an extra adenosine as a result of non-template adenylation as determined by the ESI-MS. However, the products from the B. melitensis reaction contained both non-templated adenylated strands and non-adenylated strands. A variety of adenylated and non-adenylated product patterns were observed from the various isolates and CSTs analyzed in this study. The base compositions for the forward strand of each isolate and CST in Table 6 were calculated taking into account the presence of both uracils and thiamines but for clarity, were reported as if thiamines only were incorporated. Likewise, all reported base counts reflect native products without extra adenylation.

Identical base compositions were determined at each dilution for all targets and reflected the expected composition of each isolate and its associated CST for each reaction product. All RT-PCR and reverse transcriptase RT-PCR positive reactions were detected by ESI-MS; however, there were instances of ESI-MS detection of amplicons that did not result in defined Ct values from the RT-PCR and reverse RT-PCR reactions (Table 7, R. communis isolate copy level of 1, 4/6 PCR positives vs. 6/6 MS positives; Table 8, Nipah virus CST copy level of 10, 1/6 PCR positive vs. 4/6 MS positives).

### Template Mixture Experiments

In the event of a contamination event, some level of CST would be unknowingly introduced into the isolate sample reaction. It is difficult to know from Ct values the levels of the specific CST and isolate amplicons. These mixtures, while indistinguishable by Ct, were clearly resolved in ESI-MS analysis.

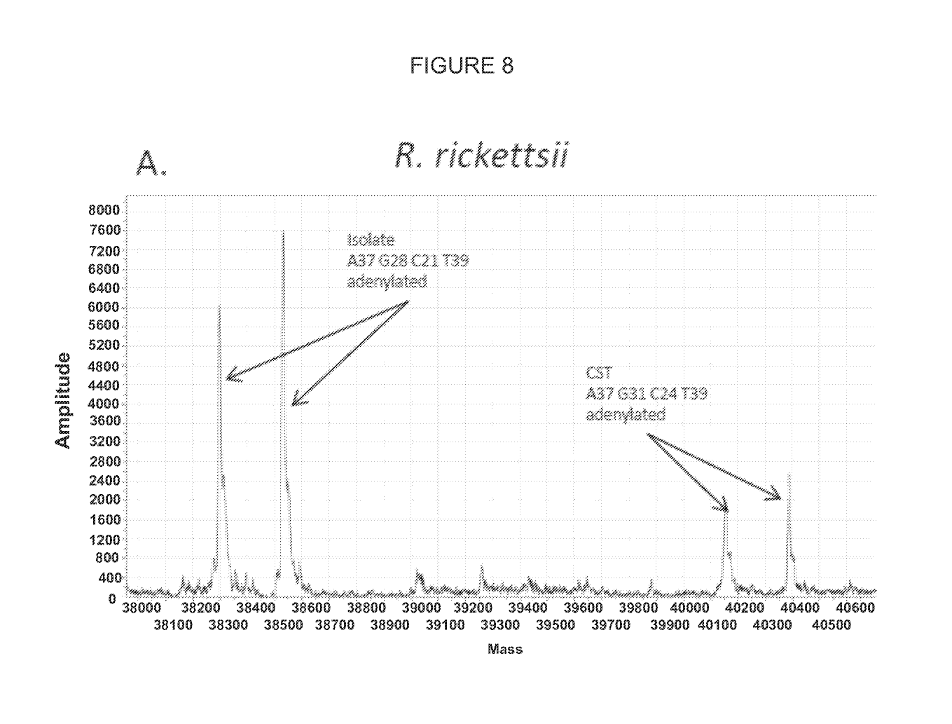

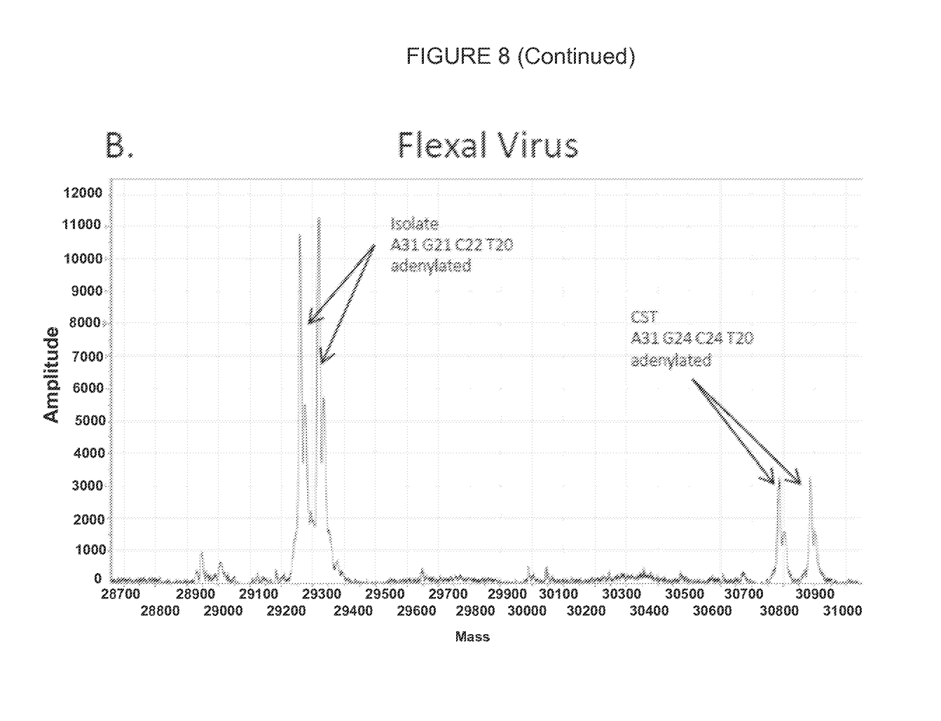

As an example of a contaminated RT-PCR reaction, 10,000 copies of Rickettsia rickettsii isolate and 100 copies of its specific CST were combined in six replicates prior to thermocycling. The combined Ct average was 33.45+/−0.10, representing both products as they are indistinguishable by this assay alone. As seen in FIG. 8a, the ESI-MS analysis clearly differentiated and individually identified the products from both templates.

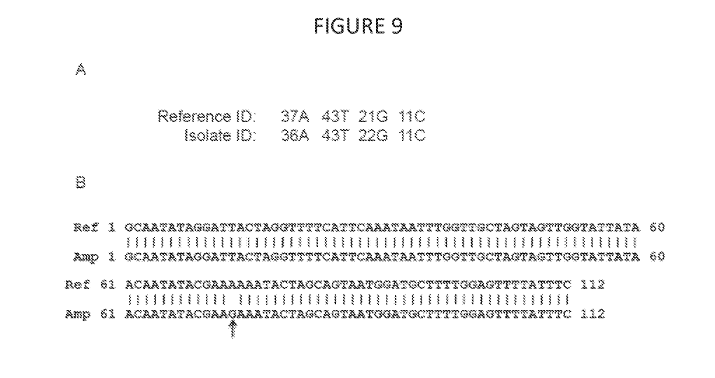

Flexal virus was chosen to provide an example of mixed templates in reverse transcriptase RT-PCR. The combined templates contained isolate at approximately 100 copies and the CST at approximately 10 copies. Their combined Ct average was 31.51+/−0.21, again representing both products. As seen in FIG. 9b, the ESI-MS analysis, again, clearly differentiated and identified the products from both templates.

### SNP Detection and Verification

The sensitivity of the ESI-MS allowed detection of a SNP between the C. botulinum F RT-PCR product and the composition reported for the reference in GenBank (Accession CP000728.1). The detected base count from the isolate nucleic acid differed from the predicted reference GenBank base count by an A-G SNP (FIG. 9A). Subsequent sequence analysis of the purified isolate amplicon DNA confirmed the A-G SNP transition (FIG. 9B).

TABLE 6

| | | Forward Strand |
|---|---|---|
| Species | Target | Mass Spec ID^{d} |
| B. melitensis | Isolate | A18 G35 C26 T20 |
| | CST | A18 G37 C28 T20 |
| F. tularensis | Isolate | A38 G15 C11 T29 |
| | CST | A38 G17 C14 T29 |
| R. communis | Isolate | A25 G21 C22 T27 |
| | CST | A25 G24 C23 T27 |
| R. prowazekii | Isolate | A33 G23 C25 T38 |
| | CST | A33 G26 C28 T38 |
| R. rickettsii | Isolate | A37 G28 C21 T39 |
| | CST | A37 G31 C24 T39 |
| R. ryphi | Isolate | A39 G20 C25 T38 |
| | CST | A39 G23 C28 T38 |
| Nipah Virus | Isolate | A36 G35 C14 T21 |
| | CST | A35 G36 C16 T21 |
| Hendra Virus | Isolate | A17 G17 C18 T17 |
| | CST | A17 G19 C21 T17 |
| Flexal Virus | Isolate | A31 G21 C22 T20 |
| | CST | A31 G24 C24 T20 |

CST = control synthetic template

TABLE 7

| | | RT-PCR | | ESI-MS |
|---|---|---|---|---|
| Target | Copies^{a} | Positives | Av Ct^{b} | Positives^{c} |
| B. melitensis | 800 | 6/6 | 30.43 ± 0.84 | 6/6 |
| Isolate | 40 | 6/6 | 34.63 ± 0.28 | 6/6 |
| | 4 | 6/6 | 38.17 ± 0.53 | 6/6 |
| | 0.4 | 2/6 | 40.25 ± 0.73 | 2/6 |
| CST | 10,000 | 6/6 | 26.46 ± 0.09 | 6/6 |
| | 1000 | 6/6 | 29.84 ± 0.09 | 6/6 |
| | 100 | 6/6 | 33.28 ± 0.05 | 6/6 |
| | 10 | 6/6 | 36.68 ± 0.49 | 6/6 |
| NTC | 0 | 0/10 | NA | 0/10 |
| F. tularensis | 6800 | 6/6 | 27.74 ± 0.20 | 6/6 |
| Isolate | 700 | 6/6 | 31.41 ± 0.46 | 6/6 |
| | 90 | 6/6 | 34.60 ± 0.30 | 6/6 |
| | 10 | 6/6 | 38.41 ± 1.0 | 6/6 |
| CST | 10,000 | 6/6 | 26.95 ± 0.44 | 6/6 |
| | 1000 | 6/6 | 30.83 ± 0.29 | 6/6 |
| | 100 | 6/6 | 34.17 ± 0.28 | 6/6 |
| | 10 | 6/6 | 38.05 ± 1.0 | 6/6 |
| NTC | 0 | 0/10 | NA | 0/10 |
| R. communis | 90 | 6/6 | 32.58 ± 0.17 | 6/6 |
| Isolate | 10 | 6/6 | 35.97 ± 0.64 | 6/6 |
| | 1 | 4/6 | 39.12 ± 0.55 | 6/6 |
| CST | 1000 | 6/6 | 29.00 ± 0.04 | 6/6 |
| | 100 | 6/6 | 32.23 ± 0.20 | 6/6 |
| | 10 | 6/6 | 35.97 ± 0.58 | 6/6 |
| NTC | 0 | 0/10 | NA | 0/10 |
| R. prowazekii | 20,000 | 6/6 | 28.82 ± 0.10 | 6/6 |
| Isolate | 2000 | 6/6 | 32.23 ± 0.11 | 6/6 |
| | 200 | 6/6 | 35.68 ± 0.22 | 6/6 |
| | 20 | 6/6 | 39.47 ± 0.50 | 6/6 |
| CST | 10,000 | 6/6 | 29.9 ± 0.10 | 6/6 |
| | 1000 | 6/6 | 33.29 ± 0.18 | 6/6 |
| | 100 | 6/6 | 36.71 ± 0.20 | 6/6 |
| | 10 | 6/6 | 40.41 ± 1.2 | 6/6 |
| NTC | 0 | 0/10 | NA | 0/10 |
| R. rickettsii | 20,000 | 6/6 | 28.89 ± 0.08 | 6/6 |
| Isolate | 2000 | 6/6 | 32.31 ± 0.10 | 6/6 |
| | 200 | 6/6 | 35.71 ± 0.20 | 6/6 |
| | 20 | 6/6 | 39.53 ± 0.64 | 6/6 |
| CST | 10,000 | 6/6 | 29.82 ± 0.09 | 6/6 |
| | 1000 | 6/6 | 33.17 ± 0.13 | 6/6 |
| | 100 | 6/6 | 36.71 ± 0.22 | 6/6 |
| | 10 | 6/6 | 40.84 ± 0.61 | 6/6 |
| NTC | 0 | 0/10 | NA | 0/10 |
| R. ryphi | 22,000 | 6/6 | 26.30 ± 0.10 | 6/6 |
| Isolate | 2200 | 6/6 | 29.63 ± 0.11 | 6/6 |
| | 220 | 6/6 | 33.03 ± 0.11 | 6/6 |
| | 20 | 6/6 | 36.58 ± 0.36 | 6/6 |
| CST | 10,000 | 6/6 | 27.39 ± 0.07 | 6/6 |
| | 1000 | 6/6 | 30.74 ± 0.09 | 6/6 |
| | 100 | 6/6 | 34.30 ± 0.35 | 6/6 |
| | 10 | 6/6 | 37.57 ± 0.43 | 6/6 |
| NTC | 0 | 0/10 | NA | 0/10 |

CST = control synthetic template
NTC = no template control
NA = Not applicable
^{a}For isolate, copy numbers are estimated from standard curve derived from CST.
^{b}Average Ct values reflect the number of positives (out of 6) as reported in the RT-PCR Positive column.
^{c}ESI-MS positives reflect the number of samples that produced clearly defined peaks on the mass spectra of the correct MW for both the forward and reverse strands with and/or without adenylation.
^{d}Native forward strand (non-adenylated) ESI-MS base composition.

TABLE 8

| | | rt RT-PCR | | ESI-MS |
|---|---|---|---|---|
| Target | Copies^{a} | Positives | Av Ct^{b} | Positives^{c} |
| Nipah Virus | 200,000 | 6/6 | 24.04 ± 0.11 | 6/6 |
| Isolate | 16,000 | 6/6 | 27.69 ± 0.05 | 6/6 |
| | 1400 | 6/6 | 31.03 ± 0.15 | 6/6 |
| | 120 | 6/6 | 34.49 ± 0.34 | 6/6 |
| CST | 100,000 | 6/6 | 25.07 ± 0.10 | 6/6 |
| | 10,000 | 6/6 | 28.41 ± 0.09 | 6/6 |
| | 1000 | 6/6 | 31.54 ± 0.15 | 6/6 |
| | 100 | 6/6 | 34.57 ± 0.10 | 6/6 |
| | 10 | 1/6 | 37.86^{e} | 4/6 |
| NTC | 0 | 0/10 | NA | 0/10 |
| Hendra Virus | 3000 | 6/6 | 28.43 ± 0.08 | 6/6 |
| Isolate | 300 | 6/6 | 31.90 ± 0.14 | 6/6 |
| | 30 | 6/6 | 35.30 ± 0.28 | 6/6 |
| | 5 | 6/6 | 37.88 ± 0.40 | 6/6 |
| CST | 10,000 | 6/6 | 26.76 ± 0.08 | 6/6 |
| | 1000 | 6/6 | 30.18 ± 0.12 | 6/6 |
| | 100 | 6/6 | 33.61 ± 0.27 | 6/6 |
| | 10 | 6/6 | 36.84 ± 0.80 | 6/6 |
| NTC | 0 | 0/10 | NA | 0/10 |
| Flexal Virus | 100,000 | 6/6 | 21.56 ± 0.10 | 6/6 |
| Isolate | 10,000 | 6/6 | 24.95 ± 0.07 | 6/6 |
| | 1,000 | 6/6 | 28.33 ± 0.17 | 6/6 |
| | 95 | 6/6 | 31.80 ± 0.08 | 6/6 |
| | 11 | 6/6 | 35.02 ± 0.30 | 6/6 |
| CST | 100,000 | 6/6 | 21.60 ± 0.16 | 6/6 |
| | 10,000 | 6/6 | 24.96 ± 0.12 | 6/6 |
| | 1000 | 6/6 | 28.34 ± 0.07 | 6/6 |
| | 100 | 6/6 | 31.62 ± 0.15 | 6/6 |
| | 10 | 6/6 | 35.19 ± 0.28 | 6/6 |
| NTC | 0 | 0/10 | NA | 0/10 |

CST = control synthetic template
NTC = no template control
NA = not applicable
^{a}For isolate, copy numbers are estimated from standard curve derived from CST.
^{b}Average Ct values reflect the number of positives (out of 6) as reported in the rt RT-PCR Positive column.
^{c}ESI-MS positives reflect the number of samples that produced clearly defined peaks on the mass spectra of the correct MW for both the forward and reverse strands with and/or without adenylation.
^{d}Reported values are for the native strand (non-adenylated).
^{e}Standard deviation not appliciable as only one replicate was detected.

Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims. All references throughout the specification are herein incorporated by reference in their entireties.

1. A method of detecting the presence of a nucleic acid in a sample comprising:
(a) enzymatically amplifying a segment of said nucleic acid to produce an amplicon comprising five or more different types of nucleotides;
(b) measuring the molecular mass of said amplicon by mass spectrometry;
(c) determining a base composition of said amplicon;
(d) detecting the presence of said nucleic acid in said sample.

2. The method of claim 1, wherein enzymatically amplifying comprises amplifying by PCR.

3. The method of claim 2, wherein amplifying by PCR comprises amplifying by RT-PCR, (rt) RT-PCR, or qPCR.

4. The method of claim 1, wherein enzymatically amplifying comprises combining said nucleic acid or said segment thereof in a reaction vessel with:
(i) a primer pair comprising a forward primer and a reverse primer,
(ii) a mixture of conventional dNTPs, wherein said mixture is lacking one dNTP selected from dATP, dCTP, dGTP, or dTTP;
(iii) a modified dNTP;
(iv) a DNA polymerase enzyme capable of incorporating said modified dNTP in place of the dNTP missing from said mixture of conventional dNTPs; and
(v) appropriate buffer, salt and pH conditions for enzymatic amplification of nucleic acid.

5. The method of claim 4, comprising a step before step (a) of treating said reaction vessel with an enzyme that cleaves DNA molecules at said modified dNTP.

6. The method of claim 4, where said dNTP missing from said mixture of conventional dNTPs is dTTP.

7. The method of claim 6, wherein said modified dNTP is dUTP.

8. The method of claim 7, comprising a step before step (a) of treating said reaction vessel with uracil N-glycosylase.

9. The method of claim 4, wherein said primers bind to conserved regions of said nucleic acid, wherein said conserved regions of said nucleic acid flank a variable region of said nucleic acid.

10. The method of claim 9, wherein the base composition of said variable region is sufficient to identify the genus, species, and/or strain of the bioagent from which said nucleic acid was obtained.

11. The method of claim 9, wherein said primers do not comprise said modified nucleotide.

12. The method of claim 11, wherein said primers comprise deoxyadenosine, deoxycytosine, deoxyguanosine, and deoxythymine.

13. The method of claim 12, wherein said amplicon comprises deoxyadenosine, deoxycytosine, deoxyguanosine, deoxythymine, and deoxyuracil.

14. The method of claim 1, wherein mass spectrometry comprises ESI-MS.

15. The method of claim 1, wherein determining a base composition of said amplicon does not comprise determining the sequential order of nucleotides in said amplicon.

16. A method of detecting the presence of a nucleic acid in a sample comprising:
(a) combining said nucleic acid or a portion thereof in a reaction vessel with:
(i) a primer pair comprising a forward primer and a reverse primer,
(ii) a mixture of conventional dNTPs, wherein said mixture is lacking one dNTP selected from dATP, dCTP, dGTP, or dTTP;
(iii) a modified dNTP;
(iv) a DNA polymerase enzyme capable of incorporating said modified dNTP in place of the dNTP missing from said mixture of conventional dNTPs; and
(v) an enzyme that cleaves DNA molecules at said modified dNTP;
(b) incubating the contents of said reaction mixture at a temperature wherein said enzyme that cleaves DNA molecules at said modified dNTP is active, but said DNA polymerase enzyme is not active, under conditions and for a time sufficient to degrade nucleic acids containing said modified dNTP;
(c) incubating the contents of said reaction mixture at a temperature wherein said DNA polymerase enzyme is active, but said enzyme that cleaves DNA molecules at said modified dNTP is not active, under conditions and for a time sufficient to amplify a segment of said nucleic acid to produce an amplicon;
(d) measuring the molecular mass of said amplicon by mass spectrometry;
(e) determining a base composition of said amplicon;
(f) detecting the presence of said nucleic acid in said sample.

17-27. (canceled)

28. The method of claim 16, wherein determining a base composition comprises correcting the molecular weight contribution of said modified dNTPs with a molecular weight contribution for a corresponding number of the dNTP missing from said mixture of conventional dNTPs.

29. The method of claim 16, wherein said enzyme that cleaves DNA molecules at said modified dNTP is active at a temperature range between 45 and 60° C.

30-36. (canceled)

37. A method of detecting the presence of a nucleic acid in a sample comprising:
(a) combining said nucleic acid or a portion thereof in a reaction vessel with:
(i) a primer pair comprising a forward primer and a reverse primer,
(ii) a mixture of conventional dNTPs, wherein said mixture is lacking one dNTP selected from dATP, dCTP, dGTP, or dTTP;
(iii) a modified dNTP;
(iv) a DNA polymerase enzyme capable of incorporating said modified dNTP in place of the dNTP missing from said mixture of conventional dNTPs, wherein said DNA polymerase enzyme is capable of catalyzing non-templated adenylation; and
(v) an enzyme that cleaves DNA molecules at said modified dNTP;
(b) incubating the contents of said reaction mixture at a temperature wherein said enzyme that cleaves DNA molecules at said modified dNTP is active, but said DNA polymerase enzyme is not active, under conditions and for a time sufficient to degrade nucleic acids containing said modified dNTP;
(c) incubating the contents of said reaction mixture at a temperature wherein said DNA polymerase enzyme is active, but said enzyme that cleaves DNA molecules at said modified dNTP is not active, under conditions and for a time sufficient to amplify a segment of said nucleic acid to produce an amplicon;
(d) measuring the molecular mass of said amplicon by mass spectrometry;
(e) determining a base composition of said amplicon by correcting for the incorporation of non-templated adenylation; and
(f) detecting the presence of said nucleic acid in said sample.

38-48. (canceled)

49. The method of claim 37, wherein determining a base composition comprises correcting the molecular weight contribution of said modified dNTPs with a molecular weight contribution for a corresponding number of the dNTP missing from said mixture of conventional dNTPs.

50-51. (canceled)
